(19) United States
(12) Patent Application Publication
(10) Pub. No.: US 2006/0073154 A1
(43) Pub. Date: Apr. 6, 2006

(54) Group a streptococci bind to mucin and human pharyngeal cells through a sialic acid-containing receptor

(75) Inventors: Patricia Ryan, New York, NY (US); Vijay Pancholi, Forest Hills, NY (US); Vincent Fischetti, West Hempstead, NY (US)

Correspondence Address: DARBY & DARBY P.C., P. O. BOX 5257, NEW YORK, NY, 10150-5257, US

(73) Assignee: The Rockfeller University

(21) Appl. No.: 10/233,074

(22) Filed: Aug. 30, 2002

Related U.S. Application Data
(60) Provisional application No. 60/317,371, filed on Sep. 4, 2001.

(51) Int. Cl. A61K 39/40; A61K 39/02; G01N 33/554; G01N 33/569; C07K 14/315

(52) U.S. Cl. 424/165.1; 514/54; 435/7.32; 514/44; 424/190.1; 530/350

(57) ABSTRACT

The present invention comprises methods of inhibiting the interaction between group A streptococci or M protein and mucin or mucin producing cells by interfering with binding of M protein to sialic acid moieties of mucin, as well as methods of identifying modulators (e.g., inhibitors) of the interaction and novel M-protein binding receptors and mucin binding receptors. Also provided are methods of treating a group A streptococcal infection by inhibiting the interaction as well as pharmaceutical compositions which may be useful for this purpose.

### PRIORITY

This application claims priority under 35 U.S.C. § 119 from provisional patent application Ser. No. 60/317,371, filed Sep. 4, 2001, which is hereby incorporated by reference in its entirety.

### GOVERMENT SUPPORT

The research leading to the present invention was supported in part by National Institutes of Health Grant No. AI 11822. Accordingly, the U.S. Government may have certain rights in the invention.

### SEQUENCE LISTING

This application contains a Sequence Listing contained on a floppy disk and hard copy.

### FIELD OF THE INVENTION

The present invention relates to methods and compositions for inhibiting binding of group A streptococci to cells that display sialic acid on their surface.

### BACKGROUND OF THE INVENTION

Group A streptococci (e.g., Streptococcus pyogenes) are the causative agents of numerous infections such as acute pharyngitis and impetigo, and are associated with the post-streptococcal sequellae, rheumatic fever and glomerulonephritis. The upper respiratory pharyngeal mucosa is the primary site of adherence and colonization by these organisms and a number of their surface proteins have been shown to be important in this process (Caparon, M., et al., Infect. Immun. 59:1811-1817, 1991; Cue, D. et al., Infect. Immun. 66:4593-4601, 1998; Cue, D. R., et al., Infect. Immun. 65:2759-2764, 1997; Fluckiger, U., et al, Infect. Immun. 66:974-979, 1999 and Hollingshead, S. K., et al., Infect. Immun. 61:2277-2283, 1993). One of these proteins, the M protein (for review see Fischetti, V. A., Clin. Microbiol. Rev. 2:285-314, 1989) is a fibrillar molecule that is considered to be the major virulence factor of S. pyogenes because it renders these organisms resistant to phagocytosis (Fischetti, V. A., Clin. Microbiol. Rev. 2:285-314, 1989; Manjula, B. N., et al., Infect. Immun. 50:610-613, 1985 and Peterson, P., et al., J. Infect. Dis. 139:575-585, 1979) and is involved in the adherence to pharyngeal tissue (Ellen, R. P., et al., Infect. Immun. 5:826-830, 1972 and Tylewska, S. K., et al., Curr. Microbiol. 16:209-216, 1988). There is a large body of work on the interaction of group A streptococci with pharyngeal epithelial cells and the virulence factors, other than the M protein, which are necessary to initiate infection (Alkan, M., et al., Infect. Immun. 18:555-557, 1977; Simpson, W. A., et al., Infect. Immun. 39:275-279, 1983; Talay, S. R., et al., Infect. Immun. 60:3837-3844, 1992 and Tylewska, S. K., et al., Curr. Microbiol. 16:209-216, 1988). Many of these reports have focused on the adherence of streptococci to various glycoproteins, including fibronectin, plasminogen and collagen (Cunningham, M. W., et al., Clin. Microbiol. Rev. 13:470-511, 2000; Fischetti, V. A., Clin. Microbiol. Rev. 2:285-314, 1989 and Ozeri, V., I., et al., Mol. Microbiol. 30:625-637, 1998). Although much is known about the streptococcal adhesins involved in these interactions, the identity of the epitope(s) on these glycoproteins that are responsible for binding is heretofore unknown.

Despite numerous reports on streptococcal adherence to various glycoproteins, no information is available on the interaction of these organisms with mucin, the major glycoprotein component of respiratory tract mucus.

By virtue of its anatomical location, the mucus layer coating all mucous membranes is the first major barrier encountered by nearly all pathogens, including group A streptococci. The function of respiratory tract mucus is the entrapment of invading microorganisms and particulate matter to prevent pulmonary infection. Mucus consists of a major glycoprotein, mucin, in addition to several other components such as serum glycoproteins, lipids and immunoglobulins (Strous, G. J., et al., Crit. Rev. Biochem. Mol. Biol. 27:57-92, 1992 and Woodward, H., B., et al., Biochem. 21:694-701, 1982). Mucins are complex, carbohydrate-rich glycoproteins secreted by mucosal and submucosal glands, and are generally subdivided into two major types, a secretory soluble type and a membrane bound type (see reviews Strous, G. J., et al., Crit. Rev. Biochem. Mol. Biol. 27:57-92, 1992 and Tabak, L., A. Annu. Rev. Physiol. 57:547-564, 1995).

Numerous bacterial pathogens such as Pseudomonas aeruginosa (Ramphal, R., et al., Infect. Immun. 55:600-603, 1987; Ramphal, R., et al., Infect. Immun. 41:339-344, 1983; Vishwanath, S., et al., Infect. Immun. 45:197-202, 1984 and Vishwanath, S., et al., Infect. Immun. 48:331-335, 1985), Pseudomonas cepacia (Sajjan, S. U., et al., Infect. Immun. 60:1434-1440, 1998), Staphylococcus aureus (Shuter, J., et al., Infect. Immun. 64:310-318, 1998) and Haemophilus influenzae (Davies, J., et al., Infect. Immun. 63:2485-2492, 1995) have been shown to bind to mucin; however the adhesins involved have not been well characterized and the actual mucin binding epitopes of these proteins have not been identified. Although S. pyogenes is a common upper respiratory tract pathogen, the details of its interaction with mucins have not been previously investigated.

Thus, there is a need in the art to better understand, and develop strategies to block bacterial binding to mucin. There is a further need to determine whether group A streptococci bind mucin, and if so, how to block such binding interactions. This invention addresses these and other needs in the art.

### SUMMARY OF THE INVENTION

The present invention comprises a method for binding group A streptococcal M protein (e.g., an isolated M protein) to sialic acid containing protein, particularly to mucin, comprising contacting the M protein with the sialic acid containing protein, particularly mucin, as well as methods of inhibiting the binding. Interaction between M protein and mucin can be prevented either by contacting the M protein or by contacting the mucin with a substance which prevents binding. In a specific embodiment of the invention, binding may be prevented by contacting M protein with sialic acid, 6′-siallyllactose, an anti-M-protein antibody (e.g, 10A11 antibody of 10B6 antibody) or transferrin or by contacting mucin with neuraminidase (e.g., C. perfringens neuraminidase) or soluble M protein or a fragment thereof (e.g, a soluble form of an N-terminal mucin binding fragment of M protein such as the N-terminal fragment of M protein which is produced by pepsin digestion of the protein). The M protein may be isolated or associated with a group A streptococcal cell, preferably strain D471, and the mucin may be isolated or associated with a membrane-bound mucin producing cell, preferably a pharyngeal cell, more preferably, strain Detroit 562.

The invention also provides methods of treating group A streptococcal infections in a patient, preferably wherein the infection site comprises membrane bound mucin-producing cells, particularly pharyngeal cells, by preventing adhesion and/or invasion of a cell at the infection site by group A streptococci, preferably by inhibiting binding between M protein and a receptor of a cell at the site (e.g., a pharyngeal cell comprising mucin). Preferably, the group A streptococcal infection is treated by administering sialic acid, 6′-sialyllactose, transferrin, neuraminidase, an anti-M-protein antibody (e.g, 10B6 antibody or 10A11 antibody), or a soluble form of M protein or a fragment thereof (e.g, a soluble form of an N-terminal mucin binding fragment of M protein such as the N-terminal fragment of M protein which is produced by pepsin digestion of the protein) to the patient. Also provided are pharmaceutical compositions comprising a pharmaceutically acceptable carrier and sialic acid, 6′-sialyllactose, transferrin, neuraminidase, an anti-M-protein antibody (e.g, 10B6 antibody or 10A11 antibody), or a soluble form of M protein or a fragment thereof (e.g, a soluble form of an N-terminal mucin binding fragment of M protein such as the N-terminal fragment of M protein which is produced by pepsin digestion of the protein). The invention also comprises vaccines which may include a pharmaceutically acceptable carrier and a group A streptococcal M protein or fragment thereof (e.g, a soluble form of an N-terminal mucin binding fragment of M protein such as the N-terminal fragment of M protein which is produced by pepsin digestion of the protein) or a nucleic acid which encodes the M protein or fragment thereof. Other substances which inhibit binding between M protein and mucin may be identified by methods of the invention. Particularly, the invention comprises methods for identifying inhibitors comprising incubating M protein or mucin with a candidate substance (e.g., an antibody, a saccharide, an enzyme, or a small molecule), contacting M protein with mucin, determining if binding occurs and selecting candidate substances which inhibit binding.

### BRIEF DESCRIPTION OF THE FIGURES

FIG. 1. Binding of group A streptococci to bovine submaxillary mucin. Adherence of S. pyogenes strain D471 to mucin and bovine serum albumin (BSA) coated wells of a microtiter plate. Wells were coated with mucin, blocked with BSA and inoculated with bacteria. The plates were incubated, washed and the bacteria were desorbed with detergent and plated. The mean±standard deviation was derived from triplicate wells in at least three independent experiments.

The inset shows the adherence of strain D471 to ¹²⁵I-mucin. Increasing concentrations of labeled mucin were added to microtiter plate wells containing immobilized heat-killed bacteria, and incubated. The wells were washed and the counts in each well (representing bound mucin) were determined in a gamma counter. Nonspecific binding was assessed as binding to BSA-coated wells and subtracted from the total binding. Means±standard deviations were obtained from triplicate wells in at least two independent experiments. The experimental details for these experiments are discussed infra (see Materials and Methods; Immobilized mucin assay and Immobilized bacterial assay).

FIG. 2. Identification of group A streptococcal cell wall proteins that bind bovine submaxillary mucin. Cell wall-associated proteins were extracted with lysin enzyme from streptococcal strains D471 and JRS75 (the isogenic M negative mutant). Extracted proteins and recombinant M protein from E. coli were separated on an 8% SDS-polyacrylamide gel and transferred to polyvinylidene difluoride (PVDF) membranes. The left panel shows an autoradiogram of a blot probed with ¹²⁵I-bovine mucin. The right panel shows a Western blot probed with the M protein-specific monoclonal antibody, 10B6. The experimental details for these experiments are discussed infra (see Materials and Methods; Identification of streptococcal cell wall proteins that bind mucin).

FIG. 3. Identification of the mucin binding region of the M protein. The N- and C-terminal portions of recombinant M protein from E. coli were isolated after pepsin digestion and separated (in duplicate) by SDS-PAGE along with undigested M protein. One gel was stained with Coomassie Brilliant Blue for visualization of total protein (left) and the other gel was electrophoretically transferred to a PVDF membrane and probed with ¹²⁵I-labeled mucin (right). The ¹²⁵I-treated membrane was analyzed autoradiographically. Arrows indicate the positions of the intact M protein (57 kDa) and the three peptide bands that represents the N-terminal portion (30 kDa) and the two C-terminal regions (15 and 12 kDa) of the pepsin-digested M protein. The experimental details for these experiments are discussed infra (see Materials and Methods; Localization of the mucin binding region of the M protein).

FIG. 4. Effect of monosaccharides on the binding of group A streptococci to bovine mucin. Streptococcal strain D471 was preincubated with N-acetylglucosamine (GlcNAc), N-acetylgalactosamine (GalNAc), fucose, galactose or N-acetylneuraminic acid (NANA, sialic acid). Bacteria were pelleted, washed three times and used to inoculate mucin coated wells. Untreated bacteria served as a control. Following incubation, adsorbed bacteria were determined. Means±standard deviations were derived from triplicate wells in at least two independent experiments. The experimental details for these experiments are discussed infra (see Materials and Methods; Effect of monosaccharides on streptococcal adherence to immobilized mucin).

FIG. 5. Effect of monosaccharides on adherence and internalization of streptococci to Detroit 562 pharyngeal cells. Streptococcal strain D471 was preincubated with 25 mM aliquots (prepared in 10 mM Tris-HCl, pH 7.4) of fucose, N-acetylgalactosamine (GalNAc), N-acetylglucosamine (GlcNAc), or N-acetylneuraminic acid (NANA). Bacteria were washed 3× and then used to infect confluent wells of Detroit 562 pharyngeal cells. Untreated bacteria served as a control. The adherence and internalization assays were performed as described in Materials and Methods; Pharyngeal cell adherence and invasion assay. Means±standard deviations were derived from triplicate wells in at least two independent experiments. The experimental details for these experiments are discussed infra (see Materials and Methods; Effect of monosaccharides on streptococcal adherence to pharyngeal cells).

FIGS. 6A-6D. Effect of sialic acid containing glycoconjugates and neuraminidase on adherence of streptococci to pharyngeal cell. (A) S. pyogenes strains D471 and the isogenic M negative strain, JRS75, were preincubated with various sialylated molecules prior to performing Detroit 562 pharyngeal cell adherence assays to determine the sialylated linkages that may play a role in adherence of the bacteria to pharyngeal cells. The streptococci were individually incubated with the following compounds: N-acetylneuraminic acid (NANA, sialic acid), 3′sialyllactose, 6′sialyllactose, fetuin, and transferrin, washed and then used in the adherence assays as described in Materials and Methods; Pharyngeal cell adherence and invasion assay. Bacteria incubated with buffer alone served as a control. The experimental details for these experiments are discussed infra (see Materials and Methods; Sialylated compounds). (B) Detroit cell monolayers were treated at confluency with Clostridium perfringens neuraminidase (C. perfringens neuraminidase cleaves, at decreasing rates, sialic acid linked α2-3, α2-6, and α2-8 (Simon, P. M., et al., Infect. Immun. 65:750-757, 1997)) to determine the effect on streptococcal adherence. Each well of cells was treated with neuraminidase. Cells incubated in buffer without enzyme served as a control. The monolayers were then washed and inoculated with strain D471 and the adherence assay was performed as described in Materials and Methods; Pharyngeal cell adherence and invasion assay. Means±standard deviations were derived from duplicate wells in at least two independent experiments. The experimental details for these experiments are discussed infra (see Materials and Methods; Neuraminidase treatment of pharyngeal cells). (C) To further verify that sialic acid and not another component of transferrin was responsible for the decrease in adherence of strain D471, 6′sialyllactose (6′SL) (which contains sialic acid linked α2-6 to galactose) was used in inhibition assays. 6′SL (1.7 mM) was able to reduce binding of this strain to pharyngeal cells by approximately 85%. (D) Pharyngeal cell monolayers were treated with C. perfringens neuraminidase prior to being inoculated with streptococcal strain D471. This treatment decreased streptococcal adherence by approximately 80%.

FIG. 7. Identification of sialylated pharyngeal cell membrane proteins which bind the streptococcal M protein. Membrane associated proteins from pharyngeal cell line Detroit 562 were extracted and solubilized as described in Materials and Methods; Preparation of Detroit 562 pharyngeal cell membranes. Extracted proteins were separated (in duplicate) on 8% SDS-polyacrylamide gels and transferred to PVDF membranes. The left panel shows a blot probed with purified M protein and the right panel shows a blot probed with 2 μg of the sialic acid-specific lectin, TML. Bound M protein and TML were detected as described in Materials and Methods. Arrows indicate the three pharyngeal cell membrane proteins that bound to both the M protein and to TML. The experimental details for these experiments are discussed infra (see Materials and Methods; Identification of sialylated proteins on pharyngeal cells which bind M protein).

### DETAILED DESCRIPTION

The present invention is based on the surprising discovery of a novel receptor-ligand interaction that mediates the interaction of group A streptococci, a highly infectious bacterium, to cells comprising sialic acid bearing proteins, preferably membrane bound mucin, on an outer surface. Specifically, mucin, a glycoprotein which is bound to the outer membrane of several cell types, particularly pharyngeal cells, has been found to mediate binding to group A streptococci via the group A streptococcal M protein. The interaction of group A streptococci and mucin is mediated by a sialic acid moiety in mucin and by an N-terminal portion of the M protein, particularly by an N-terminal 30 KDa portion of the protein. The group A streptococcal M protein also binds other sialic acid bearing proteins. Addition of free sialic acid inhibits the binding of group A streptococci to mucin containing cells. Furthermore, addition of free neuraminidase, transferrin or 6′sialyllactose has also been shown to inhibit binding.

These findings of the invention permit identification of substances that inhibit binding of group A streptococci to mucin containing cells for use in treatments of infection.

As used herein, the term “isolated” means that the referenced material is removed from the natural environment in which it is normally found. In particular, isolated biological material is free of cellular components. An isolated protein may be associated with other proteins or nucleic acids, or both, with which it associates in the cell, or with cellular membranes if it is a membrane-associated protein. An isolated organelle, cell, or tissue is removed from the anatomical site in which it is found in an organism. An isolated material may be, but need not be, purified.

The term “purified” as used herein refers to material that has been isolated under conditions that reduce or eliminate unrelated materials, i.e., contaminants. For example, a purified protein is preferably free of other proteins or nucleic acids with which it is associated in a cell.

The term “subject” or “patient” refers to any organism, preferably an animal, more preferably a mammal, and most preferably a human.

In a specific embodiment, the term “about” or “approximately” may mean within an acceptable error range for the particular measurement, e.g., 20%, preferably within 10%, and more preferably within 1%-5% of a given value or range.

Mucin producing cells may produce mucin which is secreted or mucin which remains associated with the cell (e.g., on the surface of the cell). Cells which secrete mucin may be referred to as “goblet cells.” Secretory mucin and membrane bound mucin are similar, although, distinct. The term “mucin-producing cell” refers to any cell which produces mucin, which is located anywhere in or on the cell; however, in preferred embodiments, the mucin is located on the surface of the cell. Pharyngeal cells are mucin-producing cells and pharyngeal cell strain Detroit 562 (ATCC: CCL-138) is a preferred mucin-producing cell.

The terms “sialic acid”, “N-acetylneuraminic acid” and “NANA” refer to these compounds and any other compounds or compositions of which they are a part. For example, sialic acid is a moiety of 3′-sialyllactose; therefore, the term “sialic acid” may include 3′-sialyllactose. Sialic acid refers to a family of 9 carbon monosaccharides, of which N-acetylneuraminic acid is the most common member, and is the metabolic precursor of a group of more than forty 9-carbon sugars (Varki, A., FASEB J., 11:248, 1997). The diversity of sialic acid lies in the various substitutions at different carbon positions in addition to the various linkages from carbon-2 to different underlying sugar chains. When sialic acid is a moiety of an oligosaccharide or polysaccharide, it is preferably linked to an adjacent sugar by an α2-3, α2-6 or α2-8 linkage.

Group A streptococci used in the present invention can be obtained from any source. For example, group A streptococci can be obtained from a clinical source, such as, from a patient suffering from a group A streptococcal infection. These isolation techniques are known in the art and can be used in the practice of the invention. Group A streptococci, may also be obtained commercially from the American Type Culture Collection (Rockeville, Md.). The term “group A streptococci” refers to bacteria which are a member of the genus Streptococcus (e.g. S. pyogenes) and which have the sugars N-acetylglucosamine and rhamnose associated with their cell walls. Strain D471 is an example of a group A streptococcal cell. Indeed, any strain of group A streptococci can be used in the practice of the present invention. Different strains may comprise variant M proteins which may exhibit various levels of affinity for mucin. It may be possible to identify isolated strains which do not exhibit significant affinity for mucin.

For the purposes of this application the term “adherence inhibitor” refers to substances which inhibit binding between group A streptococci or M protein and mucin or mucin containing cells.

The term “group A streptococcal infection” refers to any medical condition wherein a subject comprises one or more group A streptococcal cells in or on the subject's body.

### Molecular Biology and Expression of Group A Streptococcal M Protein

In practicing the present invention, many conventional techniques in protein chemistry, molecular biology, microbiology, recombinant DNA, and immunology, are used. Such techniques are well known and are explained fully in, for example, Sambrook et al., 1989, Molecular Cloning: A Laboratory Manual, Second Edition, Cold Spring Harbor Laboratory Press, Cold Spring Harbor, N.Y.; DNA Cloning: A Practical Approach, Volumes I and II, 1985 (D. N. Glover ed.); Oligonucleotide Synthesis, 1984 (M. L. Gait ed.); Nucleic Acid Hybridization, 1985 (Hames and Higgins); Transcription and Translation, 1984 (Hames and Higgins eds.); Animal Cell Culture, 1986 (R. I. Freshney ed.); Immobilized Cells and Enzymes, 1986 (IRL Press); Perbal, 1984, A Practical Guide to Molecular Cloning; the series, Methods in Enzymology (Academic Press, Inc.); Gene Transfer Vectors for Mammalian Cells, 1987 (J. H. Miller and M. P. Calos eds., Cold Spring Harbor Laboratory); Methods in Enzymology Vol. 154 and Vol. 155 (Wu and Grossman, and Wu, eds., respectively); Immunochemical Methods in Cell and Molecular Biology, 1987 (Mayer and Waler, eds; Academic Press, London); Scopes, 1987, Protein Purification: Principles and Practice, Second Edition (Springer-Verlag, N.Y.) and Handbook of Experimental Immunology, 1986, Volumes I-IV (Weir and Blackwell eds.).

A “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. A “recombinant DNA molecule” is a DNA molecule that has undergone a molecular biological manipulation.

A “polynucleotide” or “nucleotide sequence” is a series of nucleotide bases (also called “nucleotides”) in DNA and RNA, and means any chain of two or more nucleotides. A nucleotide sequence typically carries genetic information, including the information used by cellular machinery to make proteins.

A “coding sequence” or a sequence “encoding” an expression product, such as a RNA, polypeptide, protein, or enzyme, is a nucleotide sequence that, when expressed, results in the production of that RNA, polypeptide, protein, or enzyme, i.e., the nucleotide sequence encodes an amino acid sequence for that polypeptide, protein or enzyme. A coding sequence for a protein may include a start codon (usually ATG) and a stop codon.

The term “gene” means a DNA sequence that codes for or corresponds to a particular sequence of amino acids which comprise all or part of one or more proteins, and may or may not include regulatory DNA sequences, such as promoter sequences, that determine for example the conditions under which the gene is expressed. The transcribed region of a gene can include 5′- and 3′-untranslated regions (UTRs) and introns in addition to the translated (coding) region.

A “disrupted gene” refers to a gene, as described above, wherein a segment of DNA is inserted into the coding sequence of that gene thereby breaking the continuity, and possibly deleting a portion of the coding sequence.

A gene is inactivated when it is modified or acted on in such a way as to prevent the expression of a product which is as functional as the product from the same, unmodified gene.

A protein is inactivated when it is modified or acted on in such a way so as to prevent that protein from functioning at the same capacity as the same protein which has not been modified or acted on.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

As used herein, the terms “polypeptide” and “protein” may be used interchangeably to refer to a gene product or corresponding synthetic product. In specific embodiments, polypeptide or protein refers to a group A streptococcal M protein. The term “protein” may also refer specifically to the polypeptide as expressed in cells. A peptide is generally a fragment of a polypeptide, e.g., of about six or more amino acid residues.

The BLAST algorithm (e.g., BLASTN or BLASTP) refers to a sequence comparison algorithm which is known to those of ordinary skill in the art and may be accessed an any of several locations including the NCBI website. The following references discuss the BLAST algorithms in detail and are herein incorporated by reference: BLAST ALGORITHMS: Altschul, S. F., et al., J. Mol. Biol. 215:403-410,1990; Gish, W., et al., Nature Genet. 3:266-272, 1993; Madden, T. L., et al., Meth. Enzymol. 266:131-141,1996; Altschul, S. F., et al., Nucleic Acids Res. 25:3389-3402,1997; Zhang, J., et al., Genome Res. 7:649-656,1997; Wootton, J. C., et al., Comput. Chem. 17:149-163,1993; Hancock, J. M., et al., Comput. Appl. Biosci. 10:67-70, 1994; ALIGNMENT SCORING SYSTEMS: Dayhoff, M. O., Schwartz, R. M. & Orcutt, B. C. (1978) “A model of evolutionary change in proteins.” In Atlas of Protein Sequence and Structure, vol. 5, suppl. 3. M. O. Dayhoff (ed.), pp. 345-352, Natl. Biomed. Res. Found., Washington, DC; Schwartz, R. M. & Dayhoff, M. O. (1978) “Matrices for detecting distant relationships.” In Atlas of Protein Sequence and Structure, vol. 5, suppl. 3. M. O. Dayhoff (ed.), pp. 353-358, Natl. Biomed. Res. Found., Washington, DC; Altschul, S. F., J. Mol. Biol. 219:555-565,1991; States, D. J., et al., Methods 3:66-70, 1991; Henikoff, S, et al., Proc. Natl. Acad. Sci. USA 89:10915-10919, 1992; Altschul, S. F., J. Mol. Evol. 36:290-300, 1993; ALIGNMENT STATISTICS: Karlin, S., et al., Proc. Natl. Acad. Sci. USA 87:2264-2268, 1990; Karlin, S., et al., Proc. Natl. Acad. Sci. USA 90:5873-5877, 1993; Dembo, A., et al., Ann. Prob. 22:2022-2039,1994 and Altschul, S. F. (1997) “Evaluating the statistical significance of multiple distinct local alignments.” In Theoretical and Computational Methods in Genome Research (S. Suhai, ed.), pp. 1-14, Plenum, N.Y.

The term “identity” refers to exact matches between amino acid sequences in polypeptides which are being compared or between nucleotide sequences in nucleic acids which are being compared, for example, with a BLASTP algorithm or a BLASTN algorithm, respectively. As used herein, the term “sequence similarity”, “similarity”, “sequence homology” or “homology” refers to both the number of exact matches and conserved matches between the amino acid sequences of two proteins which can also be determined by using the BLASTP algorithm. A conserved match is a match between two amino acids which are of similar biochemical classification and/or biochemical properties. For example, in the context of a protein sequence comparison, a match of one amino acid with a hydrophobic side group with a different amino acid with a hydophobic side group would be considered a conserved match. Non-limiting examples of biochemical classes which are generally known by those skilled in the art are as follows: hydrophobic (valine, leucine, isoleucine, methionine, phenylalanine, tryptophan, alanine, proline); hydrophilic (histidine, lysine, arginine, glutamic acid, aspartic acid, cysteine, asparagine, glutamine, threonine, tyrosine, serine, glycine); no charge/hydrophilic (cysteine, asparagine, glutamine, threonine, tyrosine, serine, glycine); aromatic (tryptophan, tyrosine, phenylalanine); negatively charged/hydrophilic (aspartic acid, glutamic acid); positively charged/hydrophilic (histidine, lysine, arginine).

For the purposes of the present invention “M protein”, “M” and “group A streptococcal M protein” refers to any M protein gene or polypeptide from any group A streptococcal cell. Preferably, the amino acid sequence of group A streptococcal M protein is S. pyogenes M6 protein and is set forth in SEQ ID NO: 1. A nucleotide sequence encoding a preferred group A streptococcal M protein, the S. pyogenes emm6 gene, is set forth in SEQ ID NO: 2. The Genbank Accession No. for the S. pyogenes emm6 gene and M6 protein is M11338. Variants of M protein comprise polypeptides comprising at least about 70% identity or homology to a reference amino acid sequence set forth in SEQ ID NO: 1, wherein identity or homology is determined using the BLASTP algorithm, wherein parameters of the algorithm are selected to give the largest match between the sequences tested, over the entire length of the reference sequence. Variants of the gene which encodes M protein comprise nucleic acids comprising at least about 70% identity to a reference nucleotide sequence set forth in SEQ ID NO: 2, wherein identity is determined using the BLASTN algorithm, wherein parameters of the algorithm are selected to give the largest match between the sequences tested, over the entire length of the reference sequence. Functionally conserved variants of M protein refers to variants of M protein comprising mucin binding activity or mucin-producing cell binding activity or any other activity or property possessed by a group A streptococcal M protein.

The nucleotide sequence coding for M protein, or antigenic fragment, derivative or analog thereof, or a functionally active derivative, including a chimeric protein, thereof, can be inserted into an appropriate expression vector, i.e., a vector which contains the necessary elements for the transcription and translation of the inserted protein-coding sequence. Thus, the nucleic acid encoding M protein of the invention may be operationally associated with a promoter in an expression vector. Both cDNA and genomic sequences can be cloned and expressed under control of such regulatory sequences. An expression vector also preferably includes a replication origin.

M protein may also be expressed recombinantly in any suitable host-vector system. Potential host-vector systems include but are not limited to mammalian cell systems infected with virus (e.g., vaccinia virus, adenovirus, adeno-associated virus, herpes virus, etc.), or with non-viral nucleic acid vectors (e.g., expression plasmids and the like); insect cell systems infected with virus (e.g., baculovirus). Bacterial expression systems can also be used. The expression elements of vectors vary in their strengths and specificities. Depending on the host-vector system utilized, any one of a number of suitable transcription and translation elements may be used. A preferred expression host is a bacterial cell (e.g., E. coli).

Several commercially available bacterial host-vector systems are available for production and purification of M protein from bacteria. For example, the M protein gene may be introduced into a plasmid and functionally associated with a T7 RNA polymerase promoter (e.g., a pET based plasmid). The plasmid may then be transformed into an E. coli strain, such as BL21 DE3 which expresses T7 RNA polymerase when induced with lactose and lactose analogues such as isopropyl-β-D-thiogalactoside (IPTG). The M protein gene may also be introduced into a plasmid and functionally associated with a tac promoter. In a bacterial cell, tac transcription is induced in the presence of IPTG.

Expression of M protein may also be controlled by any eukaryotic promoter/enhancer element known in the art, but these regulatory elements must be functional in the eukaryotic host selected for expression. Promoters which may be used to control M protein gene expression in eukaryotic cells include, but are not limited to, cytomegalovirus (CMV) promoter, the SV40 early promoter region (Benoist, et al., Nature 290:304-310, 1981), the promoter contained in the 3′ long terminal repeat of Rous sarcoma virus (Yamamoto, et al., Cell 22:787-797, 1980), the herpes thymidine kinase promoter (Wagner, et al., Proc. Natl. Acad. Sci. U.S.A. 78:1441-1445, 1981), the regulatory sequences ofthe metallothionein gene (Brinster, et al., Nature 296:39-42, 1982); promoter elements from yeast or other fungi such as the Gal 4 promoter, the ADC (alcohol dehydrogenase) promoter, PGK (phosphoglycerol kinase) promoter, alkaline phosphatase promoter; and the animal transcriptional control regions, which exhibit tissue specificity and have been utilized in transgenic animals.

Vectors are introduced into the desired host cells by methods known in the art, e.g., transfection, electroporation, microinjection, transduction, cell fusion, DEAE dextran, calcium phosphate precipitation, lipofection (lysosome fusion), use of a gene gun, or a DNA vector transporter (see, e.g., Wu, et al., J. Biol. Chem. 267:963-967, 1992; Wu, et al., J. Biol. Chem. 263:14621 -14624,1988 and Hartmut, et al., Canadian Patent Application No. 2,012,311, filed Mar. 15, 1990).

To facilitate purification, M protein may be expressed with an affinity tag and purified by binding to an immobilized affinity matrix corresponding to the tag. For example, the protein may be expressed with a hexahistidine tag (e.g., in a pet-based HIS tag plasmid; Novagen, Inc., Madison, Wis.) and purified by immobilized metal affinity chromatography (e.g., immobilized Ni²⁺ or Co²⁺). M protein may also be expressed with a glutathione-S-transferase tag (e.g., in a pGEX-based plasmid; Amersham Pharmacia Biotech, Piscataway, N.J.) and purified on an immobilized glutathione column (e.g., glutathione sepharose).

Alternatively, an M protein of the invention can be isolated from cells that express the protein endogenously. Group A streptococci may be expressed in rich, non-selective liquid media, removed from the media and lysed. The cell lysate may then be fractionated with a series of steps including anion exchange chromatography, cation exchange chromatography, hydrophobic interaction chromatography and gel filtration. Fractions obtained from each step may be assayed to determine if M protein is present using any of the assays described below.

It is also possible to prepare M protein or fragments thereof using well-known techniques in peptide synthesis, including solid phase synthesis (using, e.g., BOC of FMOC chemistry), or peptide condensation techniques.

Bovine and porcine mucin is available commercially. Human mucin (secretory) can be purified from sputum via gel filtration chromatography and WGA (lectin) affinity chromatography.

Membrane bound mucin can be obtained by fractionating epithelial cells and then purifying (via gel filtration) the mucin from the proteins contained in the membrane extract.

### Screening Assays

In one embodiment, the invention provides a method for measuring interactions between group A streptococcal M protein and mucin. The assays may be adapted to methods for detecting substances (e.g., inhibitors) which modulate the interactions. The assays may also be adapted to identifying group A streptococcal receptors (other than M protein) which bind mucin as well as for identifying host cell receptors (other than mucin) which bind M protein.

Candidate substances may be screened from large libraries of synthetic or natural compounds. Numerous means are currently used for random and directed synthesis of saccharide, peptide, and nucleic acid based compounds. Synthetic compound libraries are commercially available from Maybridge Chemical Co. (Trevillet, Cornwall, UK), Comgenex (Princeton, N.J.), Brandon Associates (Merrimack, N.H.), and Microsource (New Milford, Conn.). A rare chemical library is available from Aldrich (Milwaukee, Wis.). Alternatively, libraries of natural compounds in the form of bacterial, fungal, plant and animal extracts are available from e.g. Pan Laboratories (Bothell, Wash.) or MycoSearch (NC), or are readily producible. Additionally, natural and synthetically produced libraries and compounds are readily modified through conventional chemical, physical, and biochemical means (Blondelle, et al., TIB Tech 14:60, 1996). In another aspect, synthetic libraries (Needels, et al., Proc. Natl. Acad. Sci. USA 90:10700-4, 1993; Ohlmeyer, et al., Proc. Natl. Acad. Sci. USA 90:10922-10926, 1993; Lam, et al., International Patent Publication No. WO 92/00252; Kocis, et al., International Patent Publication No. WO 94 28028) and the like, can be used to screen for ligands that effect complex formation.

Assays comprising immobilized group A streptococci or Mprotein. In a preferred embodiment, the method comprises immobilizing group A streptococci or M protein on a solid support, exposing the immobilized material to mucin, allowing a complex to form, washing away uncomplexed material and detecting the complex. The bacteria may be immobilized to any solid support by any acceptable method. In preferred embodiments, the cells are immobilized by binding to poly-L-lysine which is immobilized on a plastic microwell (e.g., Maxisorp microtiter plates; Nunc, Weisbaen, Germany). The present application also anticipates methods comprising immobilizing isolated group A streptococcal M protein on a solid support such as a membrane, preferably a nitrocellulose or nylon membrane, exposing the fractions to mucin and detecting complexes between M protein and mucin. Group A streptococci or M protein may also be immobilized to a solid support by binding to immobilized anti-M protein antibodies (e.g., monoclonal antibody 10A11 or monoclonal antibody 10B6). Complexes between mucin and M protein or group A streptococcal cells may be detected by any means. Preferably the mucin is detectably labeled, for example, with ¹²⁵I, where complex detection may be accomplished, after washing unbound mucin away from the bound group A streptococci or M protein, by counting gamma radiation. Gamma radiation may be detected by any method known in the art, such as scintillation counting or autoradiography. Mucin may also be detected by adding an anti-mucin antibody to the M protein/mucin complexes and detecting binding of the antibody (e.g, by a labeled secondary antibody) or by adding a mucin binding lectin, such as TML, and detecting TML binding.

These methods maybe conveniently adapted to detecting receptors, other than mucin, which bind to group A streptococci or M protein. For example, a candidate receptor may be exposed to immobilized group A streptococcal cells or M protein, and complexes may then be detected as discussed above.

Furthermore, the methods maybe adapted to test substances which inhibit interactions between group A streptococci or M protein and mucin or mucin containing cells. A candidate substance may be added to the above-mentioned assays and the affect of the substance on complex formation may then be evaluated. Alternatively, the candidate substance may be preincubated with either group A streptococci, M protein or mucin prior to testing for complex formation. Candidate substances which are correlated with decreased binding may be selected as potential binding inhibitors. Exposure of group A streptococci or M protein to sialic acid, transferrin or 6′ sialyllactose has been shown to inhibit complex formation with mucin containing cells. Exposure of mucin and mucin containing cells to neuraminidase (e.g., C. perfringens neuraminidase) has also been shown to inhibit complex formation.

Assays comprising immobilized mucin. The invention also comprises methods of detecting complex formation between group A streptococcal cells or M protein and mucin or mucin containing cells comprising immobilizing mucin or a mucin containing cell to a solid support, contacting the immobilized material with group A streptococcal cells or M protein and detecting complex formation. Complexes may be detected by any means known in the art. Preferably, the presence of group A streptococci in a complex may be detected, after washing unbound group A streptococci away from the mucin, by desorbing bound cells and culturing. Cells may be desorbed by any suitable means, such as exposure to detergent (e.g., Triton-X 100 or other mild detergents). The desorbed cells may be detected by any means including growth on culture media. Growth may be detected in liquid media (e.g., observation of optical density) or on solid media (e.g., observation of colony growth). The presence of M protein may be detected, after washing away unbound M protein from the immobilized mucin, by immunological methods, such as western blotting or ELISA analysis using an anti-M protein antibody (e.g., 10A11 or 10B6) or by detecting a label such as a radiolabel (¹²⁵I, ³²P, ³⁵S, ³H) or an epitope tag (glutathione-S-transferase, myc tag, FLAG tag, HA tag) fused or incorporated into the M protein.

These methods may be adapted to identifying receptors, preferably group A streptococcal receptors, other than M protein, which bind to mucin or mucin containing cells. In these embodiments, candidate receptors are contacted with the immobilized mucin and binding is detected as discussed above.

Furthermore, the methods may be adapted to test substances which inhibit interactions between group A streptococci or M protein and mucin. A candidate substance may be added to the above-mentioned assays and the affect of the substance on complex formation may then be evaluated. Alternatively, the candidate substance may be preincubated with either group A streptococci, M protein, mucin or a mucin containing cell prior to testing for complex formation. Candidate substances which are correlated with decreased binding may be selected as potential binding inhibitors. Exposure of mucin and mucin containing cells to neuraminidase (e.g., C. perfringens neuraminidase) has been shown to inhibit complex formation with group A streptococci and M protein. Exposure of group A streptococci or M protein to sialic acid, transferrin or 6′sialyllactose has also been shown to inhibit binding to mucin.

Cell adherence and invasion assay. The present invention also comprises assaying candidate substances for inhibition of adherence and invasion of cells comprising mucin on the cell surface (e.g., pharyngeal cells) by group A streptococci. The methods comprise incubating group A streptococci or mucin containing cells with a candidate substance, contacting the cells, and detecting the presence ofthe group A streptococci within the mucin containing cells. After the cells are contacted and cell invasion has taken place, bacteria which have not invaded the mucin containing cells may be eliminated by exposure to known bacterial antibiotics (e.g., penicillin). Since the antibiotics will not reach the bacteria located within the mucin containing cells, the bacteria will not be killed. After antibiotic treatment, the mucin containing cells may be removed from the antibiotic and lysed thereby liberating the enclosed bacteria. Group A streptococci within the cells may be detected by growth on bacterial media or any other method known in the art (e.g., PCR detection of group A streptococcal nucleic acids or western blot detection of group A streptococcal proteins such as M protein).

This assay may be adapted to identify candidate substances which inhibit adhesion and invasion of mucin-containing cells by group A streptococci. In these embodiments, the above-described adhesion and invasion assay is performed in the presence of a candidate substance and substances which are correlated with a decrease in adhesion and invasion are selected as potential adhesion/invasion inhibitors. Preexposure of the group A streptococci to a candidate substance or preexposure of the mucin containing cells to a candidate substance, prior to conducting the adherence and invasion assay, may be performed.

Rational design ofagonists/antagonists. Knowledge ofthe primary sequence of the COB inhibitory polypeptide fragment, and the similarity of that sequence with proteins of known function, can provide an initial clue as inhibitors or antagonists. Identification and screening of antagonists is further facilitated by determining structural features of the protein, e.g., using X-ray crystallography, neutron diffraction, nuclear magnetic resonance spectrometry, and other techniques for structure determination. These techniques provide for the rational design or identification of agonists and antagonists.

Currently several strategies have been employed to design substances which bind to and modulate activity of a target protein. The most rational and promising approach is based on 3D structure of proteins. This can be understood from the mechanism of drug-receptor binding, which is similar to inserting a key to a lock. The target acts as a lock with one or a few cavities. A candidate substance can bind to the target only if the 3D shape of the substance matches the shape of one of the cavities and there are favorable chemical interactions in the cavity. Hence given the 3D structure of a protein target, compounds can be designed to fit to a cavity, which is called docking. The best docked compounds can be used as leads to further design substances which modulate the mucin binding activity of M protein or the M protein binding activity of mucin by testing and optimizing their modulatory effect. Rapid progress in modeling techniques and computer technology have made it possible to do fast speed automated docking on computers. Newly developed softwares are capable of docking over 100,000 compounds to a protein in a week.

Direct binding assays. Candidate substances which modulate M protein may be identified with in vitro screens which test the ability of the substances to bind to the protein. This method has been published previously in U.S. Pat. Nos. 5,585,277 and 5,679,582 which are herein incorporated by reference in their entireties. A candidate antagonist for an M protein target is identified in this method by combining (incubating) the candidate substance with M protein, under conditions chosen to cause M protein to exist in an appropriate ratio of its folded and unfolded states or to cause the protein to unfold at an appropriate rate. Appropriate ratios and rates are dependent on assay conditions and are determined empirically for binding of M protein to the candidate substance. If the candidate substance binds M protein, the protein remains in its folded state (does not unfold). Thus, if M protein unfolds reversibly and the candidate substance binds to M protein, the relative amount of folded M protein is higher than is the case if the candidate substance does not bind M protein (i.e., the relative amount of folded M protein is higher in the presence of substance than in its absence). Thus, if a candidate substance binds to M protein, the ratio of folded target protein to unfolded target protein is greater than the corresponding ratio if the candidate substance does not bind M protein. If M protein unfolds irreversibly, the rate of unfolding is slower if the candidate substance binds to M protein than if it does not. After a given time of incubation, the ratio of folded M protein to unfolded M protein is greater than the corresponding ratio if the candidate substance does not bind M protein. A related method has also been published in U.S. Pat. No. 6,020,141 which is herein incorporated by reference in its entirety.

Candidate substances which exhibit evidence of binding to M protein in the above-described assays may be selected and further analyzed for M protein binding activity and the ability to inhibit M protein binding to mucin.

### Therapeutics

The present invention includes pharmaceutical compositions comprising any adherence inhibitor which inhibits binding between group A streptococci or M protein and mucin or mucin-containing cells. Adherence inhibitors may be identified by the methods described above (see Screening Assays). In preferred embodiments, adherence inhibitors include, but are not limited to, sialic acid, soluble protein containing a high density of sialic acid, a soluble form of the M protein N-terminal fragment, transferrin, 6′siallyllactose or neuraminidase. Anti-M-protein antibodies, such as 10A11 and 10B6 may also be use as inhibitors. Without being bound by theory, adherence inhibitors of the invention may treat and/or prevent infection by preventing streptococcal adherence to and/or invasion of mucin containing target cells, such as pharyngeal cells.

Therapeutic compositions of the invention may be used to treat any medical condition characterized by adherence of group A streptococci to mucin containing cells or tissue. Such conditions include streptococcal pharyngitis (strep throat), scarlet fever, impetigo, cellulitis/erysipelas, pneumonia, septic arthritis, bacteremia, sepsis, streptococcal toxic shock syndrome, necrotizing fasciitis, acute rheumatic fever and poststreptococcal glomerulonephritis. In preferred embodiments, the pharmaceutical compositions of the invention are used to treat group A streptococcal infection of pharyngeal cells.

In a specific embodiment, the invention provides an adherence inhibitor in an admixture with a pharmaceutically acceptable excipient. The phrase “pharmaceutically acceptable” refers to molecular entities and compositions that are physiologically tolerable and do not typically produce an allergic or similar untoward reaction, such as gastric upset, dizziness and the like, when administered to a human. Preferably, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which an adherence inhibitor is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in Remington's Pharmaceutical Sciences by E. W. Martin.

The invention further relates to a method for inhibiting group A streptococcal infections by administering an amount of a substance which inhibits binding of group A streptococci to mucin (e.g., on a cell comprising mucin, particularly pharyngeal cells) effective to inhibit such binding (i.e., an inhibitory amount). In a preferred embodiment, inhibition can have a therapeutic outcome wherein the inhibitor can be administered to a subject who is suspected of suffering from a group A streptococcal infection, in a therapeutically effective amount. The phrase “therapeutically effective amount” is used herein to mean an amount sufficient to reduce by at least about 5 percent, preferably by at least 50 percent, more preferably by at least 90-100 percent, also to prevent, group A streptococcal infections. Alternatively, a therapeutically effective amount is sufficient to cause an improvement in a clinically significant condition in the host.

Liquid preparations for oral administration can take the form of, for example, throat lozenges, mouth washes, injectants, aerosols, powders, pastes, gargles, solutions, syrups, elixirs, emulsions or suspensions, or they can be presented as a dry product for constitution with water or other suitable vehicle before use. Preparations for nasal administration may include injectants, aerosols, nasal sprays, nasal drops, nasal ointments, nasal washes, nasal injections, and nasal packings. Ointments for direct application to nasal tissues are further possible embodiments. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate. Of particular utility are high viscosity preparations, such as syrups, comprising an adherence inhibitor. These preparations are taken orally and, due to their viscosity, remain in the throat and oral cavity longer than preparations of a lower viscosity. A highly viscous liquid pharmaceutical preparation may be prepared using one or more of gelatin, pectin, xanthan gum, carrageenan or corn syrup to provide the desired level of thickness.

Preparations for oral administration can be suitably formulated to give controlled release of the active compound. For buccal administration, the therapeutics can take the form of tablets or lozenges formulated in conventional manner. The lozenge into which the adherence inhibitor is added may contain any or all of the following ingredients: corn starch, acacia, gum tragacanth, anethole, linseed, oleoresin, mineral oil, cellulose sugar, corn syrup, a variety of dyes, non-sugar sweeteners, flavorings, and any binders.

A chewable delivery system may be based on a nougat-type, chewy tablet. Such tablets generally employ a base of corn syrup (or a derivative). Such tablets are prepared as a confectionery, i.e., the corn syrup is cooked with water and a binder such as soy protein. One example of such a tablet is Tempo® antacid tablets, distributed by Thompson Medical Co., Inc., of West Palm Beach, Fla. Gum based formulations may also contain gum based products may contain any or all of the following ingredients: acacia, carnauba wax, citric acid, corn starch, food colorings, flavorings, non-sugar sweeteners, gelatin, glucose, glycerin, gum base, shellac, sodium saccharin, sugar, water, white wax, and cellulose and other binders.

For administration by inhalation, the therapeutics according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. Capsules and cartridges of, for example, gelatin for use in an inhaler or insufflator, can be formulated containing a powder mix of the compound and a suitable powder base such as lactose or starch.

In order to accelerate treatment of the infection, the administration of a second therapeutic agent may be an embodiment of this invention. The second therapeutic agent may be administered to the patient as a part of the adherence inhibitor composition or in the form of a separate composition which comprises only said second therapeutic agent. A non-limiting exemplary list of suitable second therapeutic agents includes antibacterial compositions such as penicillin, synthetic penicillins, bacitracin, methicillin, cephalosporin, polymyxin, cefaclor, cefadroxil, cefamandole nafate, cefazolin, cefixime, cefinetazole, cefonioid, cefoperazone, ceforanide, cefotanme, cefotaxime, cefotetan, cefoxitin, celpodoxime proxetil, ceftazidime, ceftizoxime, ceftriaxone, cefriaxone moxalactam, cefuroxime, cephalexin, cephalosporin C, cephalosporin C sodium salt, cephalothin, cephalothin sodium salt, cephapirin, cephradine, cefuroximeaxetil, dihydratecephalothin, moxalactam, loracarbef mafate, chelating agents and any combinations thereof in amounts which are effective to synergistically enhance the therapeutic effect of the lytic enzyme. Furthermore, antifungal compositions may be coadministered with the adherence inhibitor containing compositions of this invention (separately or within the same composition as the inhibitor). Such antifungal agents may include amphotericin B, carbol-fuchsin, ciclopirox, clotrimzole, econazole, haloprogin, ketoconazole, mafenide, miconazole, naftifine, nystatin, oxiconazole silver, sulfadiazine, sulconazole, terbinafine, tioconazole, tolnaftate, undecylenic acid, flucytosine, miconazole, or others.

Compositions of the present invention may further comprise antiviral agents. Such antiviral agents may include zinc containing substances, such as zinc gluconate. Furthermore anesthetic agents may be included in the compositions of the present invention. Suitable anaesthetics may include aspirin, acetaminophen, phenol, benzocaine, diphenhydramine, kaolin-pectin and xylocaine. Furthermore, decongestants and antihistamines may be included in compositions of the invention. Suitable decongestants may include pseudoephedrine, phenylpropanolamine and phenylephrine; antihistamines may include brompheniramine or chlorpheniramine.

### Vaccines

Another aspect of the invention relates to a method for inducing an immunological response in a subject, particularly a mammal which comprises inoculating the individual with M protein, or a fragment or variant thereof (e.g., a soluble, mucin-binding, 30 kDa, N-terminal fragment of M protein), adequate to produce an antibody and/or T cell immune response to protect the subject from infection, particularly bacterial infection and most particularly Steptococcal (e.g., group A Streptococcal) infection. Also provided are methods whereby such immunological response slows group A streptococcal infection. Yet another aspect of the invention relates to a method for inducing an immunological response in a subject which comprises delivering to the subject a nucleic acid vector to direct expression of M protein, or a fragment or a variant thereof, in vivo (DNA vaccine), in order to induce an immunological response, such as, to produce antibody and/or T cell immune response, including, for example, cytokine-producing T cells or cytotoxic T cells, to protect said individual from disease, whether that disease is already established within the individual or not.

M protein may be associated, for example by conjugation, with an immunogenic carrier protein. Various protein types may be employed as immunogenic material. These types include albumins, serum proteins, e.g., globulins, ocular lens proteins, lipoproteins, etc. Illustrative proteins include lipoprotein D from Haemophilus influenzae, Glutathione-S-transferase (GST), β-galactosidase, bovine serum albumin, keyhole limpet hemocyanin, egg ovalbumin, bovine γ-globulin, etc. The immunogenic carrier can also be a polysaccharide which is a high molecular weight polymer built up by repeated condensations of monosaccharides. Examples of polysaccharides are starches, glycogen, cellulose, carbohydrate gums, such as gum arabic, agar, and so forth. The polysaccharide can also contain polyamino acid residues and/or lipid residues. The attachment or conjugation of antigens can be accomplished by conventional processes, such as those described in U.S. Pat. No. 4,808,700, involving the addition of chemicals that enable the formation of covalent chemical bonds between the carrier immunogen and M protein. Alternatively, a multiple antigenic polypeptide comprising multiple copies of M protein, or an antigenically or immunologically equivalent polypeptide thereof may be sufficiently antigenic to improve immunogenicity so as to obviate the use of an immunogenic carrier.

The immunogen may comprise an adjuvant such as an aluminum compound (e.g., aluminum hydroxide), water and vegetable or mineral oil emulsions (e.g., Freund's adjuvant), liposomes, ISCOM (immunostimulating complex), water-soluble glasses, polyanions (e.g., poly A:U, dextran sulphate, lentinan) or combinations thereof.

Techniques for formulating such immunogens are well-known in the art. For example, the M protein immunogen of the present invention may be lyophilized for subsequent rehydration in an excipient such as saline or another physiological solution. In any event, the vaccine of the present invention is prepared by mixing an immunologically effective amount of M protein with an excipient in an amount resulting in the desired concentration of the immunogenically effective component of the vaccine. The amount of immunogenically effective component in the vaccine will depend on the subject to be immunized, with consideration given to the age and weight of the subject as well as the immunogenicity of the immunogenic component present in the vaccine.

DNA vaccines of the invention may comprise a polynucleotide encoding M protein which is operatively associated with a suitable promoter. Although an M protein gene may be in a linear or circular nucleic acid molecule, plasmid DNA is preferred. Viral promoters, such as the CMV or RSV promoters, may be associated with an M protein gene in a DNA vaccine; pcDNA3 (Invitrogen; Carlsbad, Calif.) contains the CMV promoter and pRc/RSV (Invitrogen; Carlsbad, Calif.) contains the RSV promoter.

Nucleic acid for injection into a subject may be prepared by any method known in the art which generates highly-purified material. For example, cesium chloride gradient or PEG precipitation purification methods are commonly known in the art. Commercially available nucleic acid purification kits also yield suitable material for use in DNA vaccines (e.g,.anion exchange columns from Qiagen; Valencia, Calif.).

DNA vaccines may be provided to a subject by any suitable means, preferably by injection, more preferably intramuscular injection.

### Antibodies to Group A Streptococcal M Protein

According to the invention, M protein polypeptide produced recombinantly or by chemical synthesis, and fragments or other derivatives or analogs thereof, including fusion proteins, may be used as an immunogen to generate antibodies that recognize the M protein polypeptide. Such antibodies include but are not limited to polyclonal, monoclonal, chimeric, single chain, Fab fragments, and an Fab expression library. The anti-M protein antibodies of the invention may be cross reactive, e.g., they may recognize M protein from strains or variants of group A streptococci. Polyclonal antibodies have greater likelihood of cross reactivity. The antibodies of the invention may also recognize only certain domains of M protein (e.g., an N-terminal domain or a C-terminal domain). Specifically, the invention comprises monoclonal antibodies 10A11 and 10B6 which recognize the amino- and carboxy-terminal regions of M protein, respectively.

Various procedures known in the art may be used for the production of polyclonal antibodies to M protein or derivatives or analogs thereof. For the production of antibody, various host animals can be immunized by injection with the M protein, or a derivative (e.g., fragment or fusion protein) thereof, including but not limited to rabbits, mice, rats, sheep, goats, etc. In one embodiment, M protein or a fragment thereof can be conjugated to an immunogenic carrier, e.g., bovine serum albumin (BSA) or keyhole limpet hemocyanin (KLH). Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

For preparation of monoclonal antibodies directed toward the M protein, or fragment, analog, or derivative thereof, any technique that provides for the production of antibody molecules by continuous cell lines in culture may be used. These include but are not limited to the hybridoma technique originally developed by Kohler, et al., Nature 256:495-497, 1975, as well as the trioma technique, the human B-cell hybridoma technique (Kozbor, et al., Immunology Today 4:72, 1983; Cote, et al., Proc. Natl. Acad. Sci. U.S.A. 80:2026-2030, 1983), and the EBV-hybridoma technique to produce human monoclonal antibodies (Cole, et al., in Monoclonal Antibodies and Cancer Therapy, Alan R. Liss, Inc., pp. 77-96, 1985). In an additional embodiment of the invention, monoclonal antibodies can be produced in germ-free animals (International Patent Publication No. WO 89/12690, published 28 Dec. 1989). In fact, according to the invention, techniques developed for the production of “chimeric antibodies” (Morrison, et al., J. Bacteriol. 159:870, 1984; Neuberger, et al., Nature 312:604-608, 1984; Takeda, et al., Nature 314:452-454, 1985) by splicing the genes from a mouse antibody molecule specific for M protein together with genes from a human antibody molecule of appropriate biological activity can be used; such antibodies are within the scope of this invention.

According to the invention, techniques described for the production of single chain antibodies (U.S. Pat. Nos. 5,476,786; 5,132,405 and 4,946,778) can be adapted to produce M protein-specific single chain antibodies. Indeed, these genes can be delivered for expression in vivo. An additional embodiment of the invention utilizes the techniques described for the construction of Fab expression libraries (Huse, et al., Science 246:1275-1281, 1989) to allow rapid and easy identification of monoclonal Fab fragments with the desired specificity for an M protein, or its derivatives, or analogs.

Antibody fragments which contain the idiotype of the antibody molecule can be generated by known techniques. For example, such fragments include but are not limited to: the F(abc/)₂ fragment which can be produced by pepsin digestion of the antibody molecule; the Fabc/ fragments which can be generated by reducing the disulfide bridges of the F(abc/)₂ fragment, and the Fab fragments which can be generated by treating the antibody molecule with papain and a reducing agent.

In the production of antibodies, screening for the desired antibody can be accomplished by techniques known in the art, e.g., radioimmunoassay, ELISA (enzyme-linked immunosorbant assay), “sandwich” immunoassays, immunoradiometric assays, gel diffusion precipitin reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention. For example, to select antibodies which recognize a specific epitope of M protein, one may assay generated hybridomas for a product which binds to an M protein fragment containing such epitope. For selection of an antibody specific to M protein from a particular species of animal, one can select on the basis of positive binding with M protein expressed by or isolated from cells of that species of animal.

The foregoing antibodies can be used in methods known in the art relating to the subcellular localization (e.g., in situ imaging) or detection (e.g., in the above-described assays) of M protein.

The present invention will be more fully explained by reference to the following examples, which are intended to be illustrative and not limiting thereof.

### EXAMPLES

### Example 1

### Binding of Streptococcal M Protein to Sialic Acid

### Materials and Methods

Bacterial strains and cell lines. Group A streptococcal strains D471 (M type 6) from the Rockefeller University culture collection, and the isogenic M-negative mutant, JRS75, (Norgren, M., et al., Infect. Immun. 57:3846-3850, 1989) were grown at 37° C. for 16 h in Todd Hewitt broth (Difco Labs, Detroit, Mich.) supplemented with 1% yeast extract. The bacterial concentration was determined spectrophotometrically, and viability counts were performed by plating on proteose peptone agar supplemented with 5% sheep's blood. An optical density of 1.0 at 660 nm corresponds to approximately 1×10⁸ CFU/ml.

The Detroit 562 (ATCC CCL-138) human pharyngeal cell line obtained from the American Type Culture Collection (Rockeville, Md.), was grown and maintained at 37° C. in 5% CO₂ in minimal essential medium (MEM, Gibco-BRL, Life Technologies, Grand Island, N.Y.) supplemented with 10% (v/v) fetal bovine serum and 1 mM sodium pyruvate as described before (Pancholi, V., et al., J. Exp. Med. 176:415-426, 1992).

Chemicals and reagents. Chemicals and reagents were obtained from the Sigma Chemical Co., (St. Louis, Mo.) unless otherwise mentioned.

Proteins and antisera. The monoclonal antibodies, 10A11 and 10B6, which are specific for the N- and C-terminal regions of the M protein, respectively, were produced as described in Jones, K. F., et al., J. Exp. Med. 161:623-628, 1985. The recombinant M protein was purified from Escherichia coli as described by Fischetti, et al., J. Exp. Med. 159:1083-1095, 1984. The N- and C-terminal portions of the recombinant M protein were isolated after pepsin digestion of the molecule as described previously (Fischetti, et al., J. Exp. Med. 159:1083-1095, 1984). A specific sialic acid binding lectin (Tritrichomonas mobiliensis, TML) and an anti-TML monoclonal antibody were obtained from Calbiochem-Novabiochem Corp. (San Diego, Calif.).

Preparation and radioiodination of mucin. Bovine submaxillary mucin (10 mg) in 0.1 M phosphate buffer saline (PBS, pH 7.4) was applied to a Sephadex G-200 column (PD-10, 9.1 ml bed volume, Pharmacia LKB Biotechnology, Inc., Piscataway, N.J.) equilibrated in 0.1 M PBS to remove low molecular weight contaminants. The proteins in the void volume of the column (2.5 ml), containing the high molecular weight mucins, were collected. The protein content of the fractions was determined with the BioRad (Hercules, Calif.) protein assay reagent, using the procedure outlined by the manufacturer and bovine serum albumin as the standard. Fractions of the void volume containing the highest protein concentration were radioiodinated with Na¹²⁵I (17 Ci/mg, NEN Life Science Products, Boston, Mass.) by the chloramine-T method, using Iodobeads (Pierce Chemical Co., Rockford, Ill.) as described (Pancholi, V., et al., J. Exp. Med. 176:415-426, 1992). Labeled proteins were separated from free iodine by passage over a Sephadex G-25 column (PD-10; Pharmacia) equilibrated in 0.1 M PBS, pH 6.5. The protein concentration was determined as described above. Purity of the labeled mucin was confirmed by autoradiography of SDS-polyacrylamide gels. After labeling, the specific activity of the mucin sample was 1.5×10⁵ cpm/μg.

Solid-phase mucin binding assay. To assess the ability of group A streptococci to bind to bovine submaxillary mucin, two solid phase assays were performed.

i) Immobilized mucin assay. A modified solid-phase mucin binding assay was performed with ¹²⁵I labeled bovine mucin as described previously (Vishwanath, S., et al., Infect. Immun. 45:197-202, 1984 ). Preliminary experiments revealed that 400 ng/well of the iodinated mucin was the optimum concentration needed to coat the wells of Maxisorp plates with BreakApart modules (Nunc, Naperville, Ill.). To ensure that all of the sites were bound, 1 μg of the partially purified bovine mucin was added to the wells, and the plate was incubated for 24 h at 37° C. Overnight bacterial cultures of D471 were pelleted by centrifugation (3000 rpm for 10 min at 4° C.), washed 2× in PBS and adjusted to an O.D. of 1.0 at 660 nm. Triplicate wells were inoculated with 5×10⁶ bacteria in a volume of 100 μl. Wells that contained only bovine serum albumin served as controls. The microtiter plates were incubated for 3 h at 37° C., and then washed 8 to 10 times with sterile PBS. Bound bacteria were desorbed with 250 μl of 0.5% Triton-X 100 in sterile PBS for 30 min at room temperature and plated on blood agar. The plates were incubated at 37° C. for 12-14 h and the colonies were counted.

ii) Immobilized bacterial assay. An overnight culture of strain D471 was adjusted to an O.D. of 1.0 at 660 nm, washed twice in PBS and heat killed at 55° C. for 4 h. Poly-L-lysine (PL) was prepared in PBS (100 μg/ml) and 100 μl aliquots were added to the wells of Maxisorp microtiter plates with BreakApart modules and incubated for lh at room temperature. The PL was aspirated, the wells were washed 3 times with PBS and the heat killed bacteria were added (5×10⁶ CFU in a volume of 50 μl) followed by the addition of 50 μl of 2% gluteraldehyde (in PBS). Following a 20 minute incubation period at room temperature, the plates were centrifuged (1500×g) for 20 min and the wells washed twice with sterile PBS. One hundred microliters of 0.1 M lysine solution was added to each well of the plate, and incubated at room temperature for 1 h to block excess gluteraldehyde sites. The wells were again washed with PBS and residual protein binding sites were blocked with 2% BSA in 10 mM Tris-HCl overnight at 4° C. Various concentrations of ¹²⁵I-bovine mucin were added to the wells and the plates were incubated for 4 h at room temperature. All concentrations of mucin were tested in triplicate wells. Wells that did not contain immobilized bacteria served as a control. Following this incubation, the wells were washed three times with PBS and the radioactive counts in both the wash buffer (representing free mucin) and the wells (representing bound mucin) were determined in a gamma counter.

Identification of streptococcal cell wall proteins that bind mucin. Crude extracts of the cell walls of strains D471 and JRS75 were prepared using the amidase enzyme lysin as described previously (Fischetti, V., et al., Physical, chemical and biological properties of type 6 M-protein extracted with purified streptococcal phage-associated lysin, p. 26-37. In M. J. Haverkom (ed.), Streptococcal Disease and the Community. Excerpta Medica, Amsterdam, 1974 ). All extracts were prepared in 30% raffinose to stabilize the protoplasts after the cell walls were removed. Proteins in the streptococcal cell wall extracts were subjected to electrophoresis and Western blotting techniques as described before (Ryan, P. A, et al., J. Bacteriol. 179:2551-2556, 1997). The streptococcal cell wall extracts (40 μg total protein/strain) and the recombinant M6 protein from E. coli (2 μg) were separated (in triplicate) on 8% SDS-polyacrylamide gels and were either visualized by Coomassie stain or transferred electrophoretically to polyvinylidine difluoride membranes (PVDF, Immobilon P; Millipore Corp., Bedford, Mass.). Blots were probed with radiolabeled mucin as described before (Pancholi, V., et al., J. Biol. Chem. 273:14503-14515, 1998). Duplicate membranes to be probed with antibodies were first blocked with BSA (3% in PBS) and then incubated with the anti-M protein-specific antibody, 10B6 (Jones, K. F., et al., J. Exp. Med. 161:623-628, 1985). Bound antibody was visualized with alkaline phosphatase-conjugated sheep anti-mouse immunoglobulin G, followed by the substrate 4-nitroblue tetrazolium chloride (NBT) and 5-bromo-4-chloro-3-indolyl phosphate (BCIP).

Localization of the mucin binding region of the M protein. Recombinant M protein from E. coli (5 μg) was digested with pepsin at pH 5.9 for 30 min., separated by SDS-PAGE, stained with Coomassie blue or electroblotted onto a PVDF membrane. The electroblotted membranes were probed with ¹²⁵I-bovine mucin in TBS-Tween-20 for 4 h at 30° C. Following binding with ¹²⁵I-bovine mucin, the membrane was washed 4 times (15 min/wash) in TBS-Tween-20 and autoradiographed for 12 h at −80° C. Untreated M protein (2 μg) from E. coli was considered as a control.

In a second set of experiments, the N- and C-terminal portions of the recombinant M6 protein from E. coli were isolated after pepsin digestion, separated (in duplicate) by SDS-PAGE and electrophoretically transferred to PVDF membranes. One set of Western blots was probed with MAb 10A11 (reactive with the N-terminal portion of the M protein) or MAb 10B6 (reactive with the C-terminal portion of the M protein) (Jones, K. F., et al., J. Exp. Med. 161:623-628, 1985) and developed as described above.

Identification of the component of mucin to which streptococci bind. To determine if any of the individual sugar components of mucin were important in the adherence of streptococci, the five monosaccharide constituents of the oligosaccharide side chains of mucin, N-acetylglucosamine (GlcNAc), N-acetylgalactosamine (GalNAc), N-acetylneuraminic acid (sialic acid, NANA), D-galactose and L-fucose (Strous, G. J., et al., Crit. Rev. Biochem. Mol. Biol. 27:57-92, 1992 and Woodward, H., et al., Biochem. 21:694-701, 1982), were used in a solid phase assay.

Wells of microtiter plates were coated with bovine mucin as described above. Fifty millimolar solutions of GlcNAc, GalNAc, galactose, fucose, and sialic acid were prepared in PBS. Suspensions (500 μl) of strain D471 prepared in either PBS or Tris-HCl were mixed with an equal volume of the individual sugar solutions to yield 5×10⁷ CFU/ml in 25 mM sugar solutions. Bacteria added to PBS or Tris-HCl without sugar served as the control. The mixtures were incubated for 30 min at 37° C. to allow bacteria to bind to the monosaccharide. The bacteria were then pelleted by centrifugation, washed three times and resuspended to the original volume in the same buffer. Samples (100 μl) of each bacteria-buffer or bacteria-sugar suspension were added to mucin coated wells to yield a final concentration of approximately 5×10⁶ CFU/well. The mucin adherence assay was then continued as described above.

Pharyngeal cell adherence and invasion assay. Streptococcal adherence to and invasion of the human pharyngeal cell line, Detroit 562, was assayed by modification of a procedure described previously (Talbot, U. M., et al., Infect. Immun. 64:3772-3777, 1996). Overnight cultures of streptococci were pelleted by centrifugation and washed twice in sterile PBS. The cultures were resuspended in MEM and diluted to a final concentration of 5×10⁷ CFU/ml, and 1 ml aliquots were inoculated into each well containing washed Detroit cell monolayers. At least 3 wells were used for each bacterial strain or culture condition. Following a 3 h incubation period with the bacteria at 37° C., the monolayers were washed three times in PBS.

For the bacterial invasion assay, 1 ml of MEM supplemented with penicillin (10 μg/ml) and gentamicin (200 μg/ml) was added to each well (to kill extracellular bacteria) and incubated for 1 h at 37° C. For wells used to determine total numbers of adherent and invasive bacteria, MEM without antibiotics was added to each well and incubated for an additional hour. Following the incubation, the pharyngeal cells were detached from the wells by the addition of 100 μl of 0.025% trypsin-0.02% EDTA and lysed with 400 μl of 0.025% Triton X-100. The lysates were diluted appropriately and plated on blood agar. The total number of adherent streptococci was calculated as the difference between the total number of adherent and invasive bacteria and the number of invasive bacteria alone.

Effect of monosaccharides on streptococcal adherence to immobilized mucin. Streptococcal strain D471 (5×10⁷ CFU) was preincubated with 25 mM aliquots of N-acetylglucosamine (GlcNAc), N-acetylgalactosamine (GalNAc), fucose, galactose or N-acetylneuraminic acid (NANA, sialic acid) for 30 min at 37° C. Bacteria were pelleted, washed three times and used to inoculate mucin coated wells (1 μg mucin/well inoculated with 5×10⁶ CFU/well). Untreated bacteria served as a control. Desorbed bacteria were plated on agar and counted once colonies formed. Means±standard deviations were derived from triplicate wells in at least two independent experiments.

Effect of monosaccharides on streptococcal adherence to pharyngeal cells. The effect of various monosaccharides on streptococcal adherence to pharyngeal cell monolayers was assessed in inhibition assays. Solutions (50 mM) of GlcNAc, GalNAc, fucose and sialic acid were prepared in 10 mM Tris-HCl buffered to pH 6.9 (Vishwanath, S., et al., Infect. Immun. 48:331-335, 1985). Suspensions (500 μl) of strains D471 and JRS75 prepared in Tris-HCl were mixed with an equal volume of the individual sugar solutions to yield 5×10⁷ CFU/ml in 25 mM sugar solutions. Bacteria added to PBS or Tris-HCl without sugar served as the control. Following preincubation with the individual sugars, the bacteria were washed, resuspended in MEM and added to monolayers of pharyngeal cells (5×10⁷ CFU/well). The adherence assay was continued as described above.

Sialylated compounds. The sialylated glycoproteins, fetuin and transferrin, as well as NeuAcα2-3Galβ1-4Glc (3′sialyllactose; 3′SL), and NeuACα2-6Galβ1-4Glc (6′sialyllactose; 6′SL) were tested in inhibition assays to determine the effect of each compound on streptococcal binding to pharyngeal cells. The streptococcal strains D471 and JRS75 (5×10⁷ CFU) were separately incubated (in a final volume of 1 ml) with sialic acid (25 or 2 mM), 3′SL (1.7 mM), 6′SL (1.7 mM), fetuin (1.8 mM) or transferrin (5 mg) (all prepared in 10 mM Tris-HCl HCl, pH 7.4) for 30 min at 37° C. Following this treatment, the bacteria were washed twice, resuspended in MEM and added to the pharyngeal cell monolayers. Bacteria treated with Tris-HCl (10 mM, pH 7.4) served as control. An aliquot of each of the treated bacterial samples was plated onto blood agar to ensure that the treatment had no effect on the viability of organism.

Neuraminidase treatment ofpharyngeal cells. Confluent monolayers of Detroit 562 pharyngeal cells (in 24-well tissue culture plates) were washed three times with PBS and then treated with 1U of Clostridium perfringens neuraminidase (Sigma) in 50 mM Na acetate (pH 5.5) buffer containing 5 mM CaCl₂. Cells incubated in buffer alone served as control. Cells were incubated for 30 min at 37° C., washed three times with PBS and used in the adherence assay with strain D471 (5×10⁷ CFU/well) as described above.

Preparation of Detroit 562 pharyngeal cell membranes. Detroit 562 cells were grown to confluence and washed extensively with PBS. The adherent cells were scraped with a disposable rubber cell scraper into PBS, pelleted by centrifugation for 10 min at 3000 rpm and resuspended in lysis buffer (10 mM NaH₂PO₄, pH 8, 5 mM EDTA and 1 mM PMSF). Cells were disrupted by sonication (5 sec pulses for 2 min) and cellular debris was removed by centrifugation (2000×g for 10 min). The membrane fraction was pelleted by ultracentrifugation at 100,000×g (60 min at 4° C.) and then resuspended in 0.2% Triton-X 100 and stored at −20° C. until further use.

Identification of sialylatedproteins on pharyngeal cells which bind M protein. The sialic acid binding lectin, TML, was employed to identify the pharyngeal cell membrane proteins that contain sialic acid. Pharyngeal cell membrane proteins (50 μg total protein/lane) were separated by SDS-PAGE, blotted in duplicate to PVDF membranes and were separately probed with the M protein (25 μg) and TML (1-2 μg) for 2 h at 37° C. and then washed 3× with PBS. Bound M protein and lectin were detected by incubating the blots with either the M protein specific monoclonal antibody 10B6, or with the α-TML monoclonal antibody, respectively, for 2 h at 37° C. After washing, bound antibody was visualized using alkaline phosphatase conjugated secondary antibodies and the NBT/BCIP substrate system described above.

Statistical Analysis. Differences between groups in each experiment were tested by Student's t-test. The results are expressed as means±standard deviation.

### Results

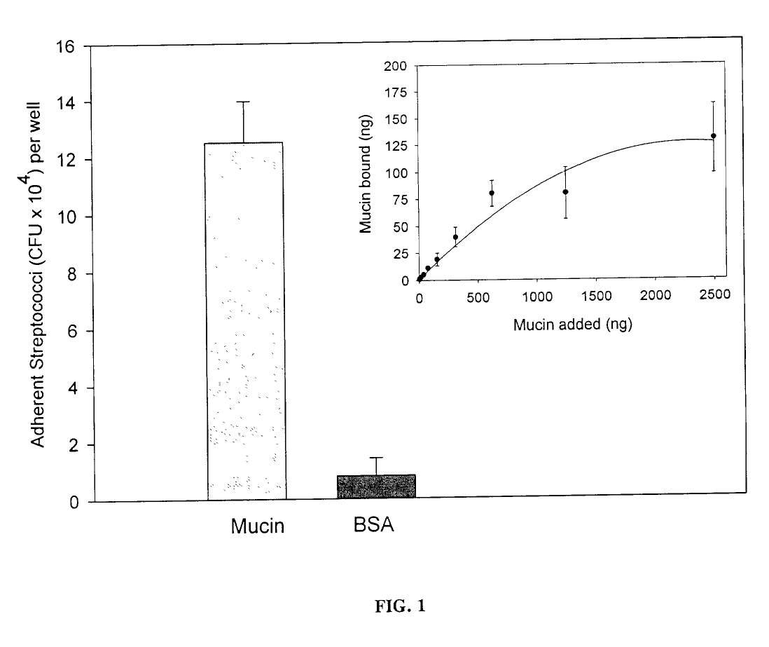

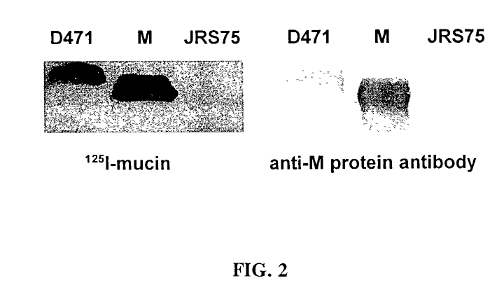

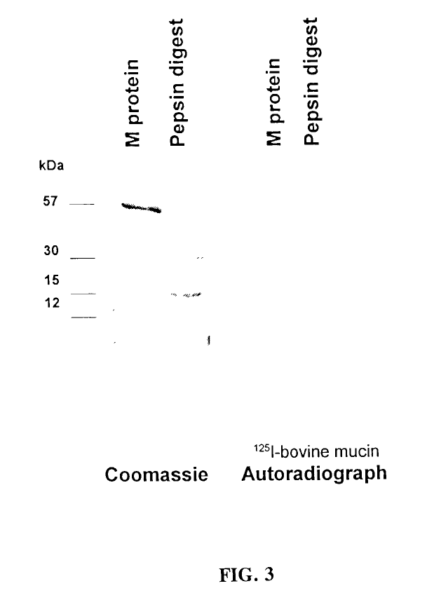

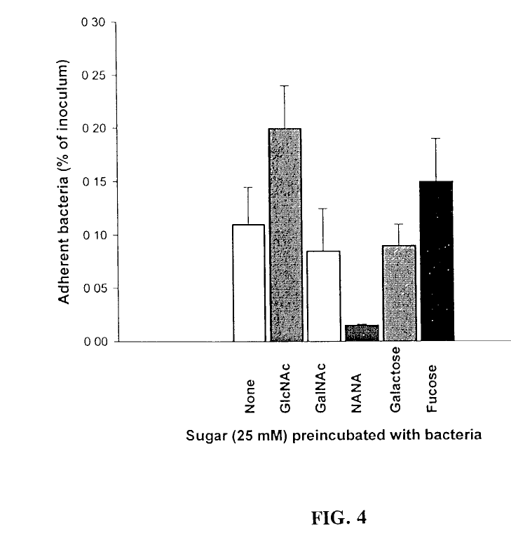

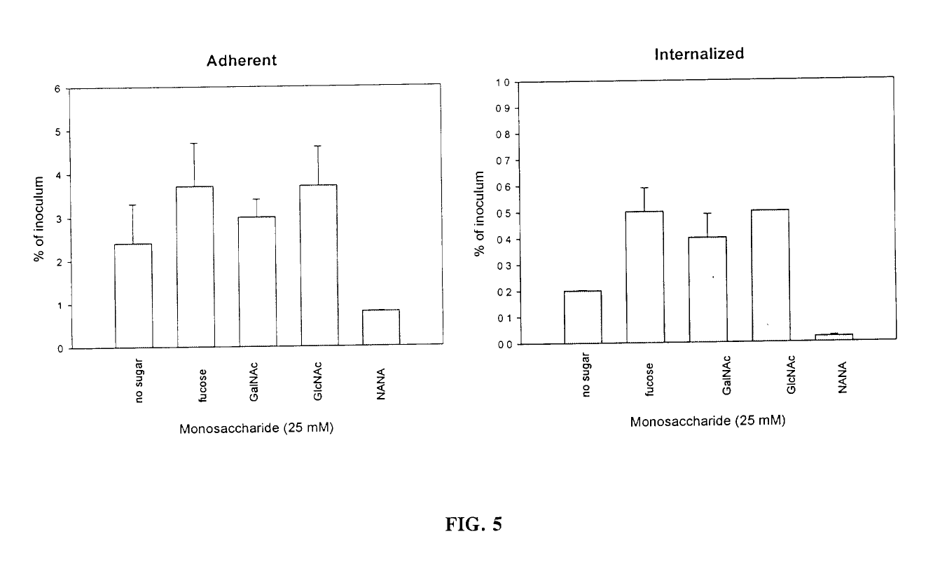

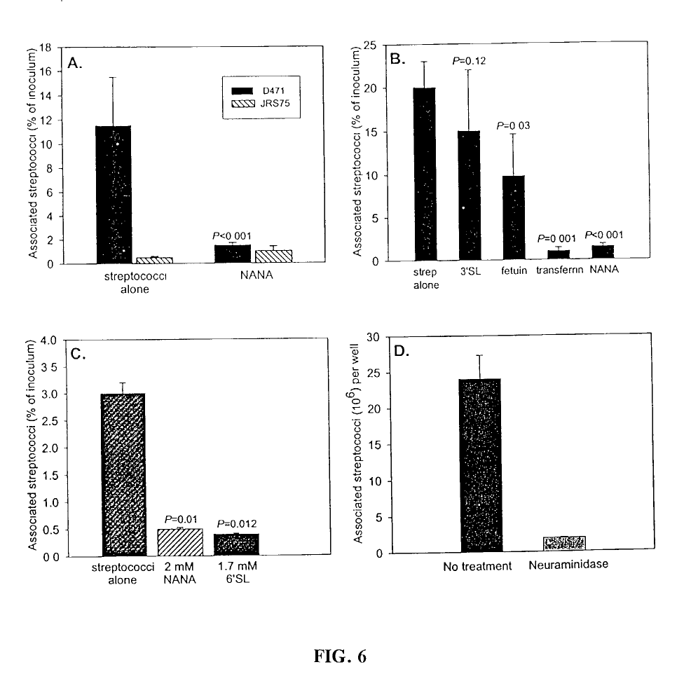

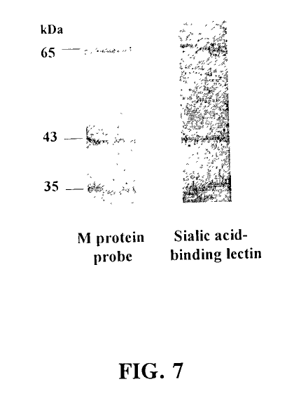

Binding of whole streptococci to mucin. The ability of S. pyogenes strain D471 to adhere to bovine mucin was examined in a solid phase assay using mucin-coated microtiter wells. As shown in FIG. 1, the binding of strain D471 to bovine mucin was significantly higher than to bovine serum albumin. To determine the specific binding activity of streptococci to mucin, a quantitative solid phase assay was employed, in which strain D471 (M+) was first immobilized to the microtiter wells and then various concentrations of radiolabeled mucin were added. As can be seen, this strain was able to bind mucin in a dose dependent manner (FIG. 1, inset).

Identification of a binding protein in streptococcal cell wall extracts. To determine which proteins on the surface of group A streptococci are capable of binding to mucin, cell wall-associated proteins from streptococcal strain D471, which were released after lysin digestion, were probed with radiolabeled bovine mucin. This analysis revealed two proteins (57 and 40 kDa) that bound mucin. The size and migration pattern of the 57 kDa protein suggested that it might be streptococcal M protein, and therefore an M-protein specific antibody and the recombinant M protein were included in the analysis. A blot of cell wall extracted proteins probed with the M protein-specific monoclonal antibody, 10B6, confirmed the identity of the 57 kDa protein as the M protein (FIG. 2). In addition, the cell wall proteins extracted from the isogenic—negative mutant, JRS75, subjected to the same analysis, did not contain the 57 kDa protein present in the extracts of strain D471, further confirming the identity of the 57 kDa as the M protein.

Localization of the mucin binding region in the Mprotein. To determine if the mucin-binding region of the M protein could be localized to the N- or C-terminal half of the molecule, the recombinant M6 molecule was digested with the enzyme pepsin. At suboptimal pH, pepsin cleaves the molecule twice, once around its center and once within the C-terminal domain (Fischetti, V. A., et al., Proteins: Struct. Func. Genet. 3:60-69, 1988). The resulting three fragments, which represent essentially the intact N-terminal half and two C-terminal segments of the protein (Fischetti, V. A., et al., Infect. Immun. 63:149-153, 1995), were separated by SDS-PAGE, and analyzed by Western blot to determine which fragment bound radiolabeled mucin. The binding was clearly localized only to the N-terminal 30 kDa fragment (FIG. 3), which was further verified by the N-terminal specific monoclonal antibody 10A11 (Jones, K. F., et al., J. Exp. Med. 161:623-628, 1985) (not shown).

Effect of sugars on streptococcal adherence. To determine if a monosaccharide component of mucin was the M protein receptor, the sugars found in mucin, GlcNAc, GalNAc, galactose, fucose and N-acetylneuraminic acid (NANA, sialic acid) were used in inhibition experiments. Pretreating strain D471 with sialic acid reduced adherence to bovine mucin by 80% (P=0.04) when compared to the untreated bacterial control (FIG. 4), clearly indicating that sialic acid is a constituent of the receptor on mucin for the M protein.

GalNAc, galactose and fucose did not significantly affect adherence (P=0.36, 0.75, and 0.67, respectively). However, when the bacteria were pretreated with GlcNAc, the number of adherent bacteria doubled as compared to untreated bacteria (P=0.3), suggesting that GlcNAc may act as a bridge between the organism and mucin, thereby enhancing adherence. Further investigation is necessary to confirm this hypothesis.

These results also indicate that sialic acid inhibited adherence by binding to the bacterial adhesin(s) rather than to the receptors on mucin, since the unbound sugar was subsequently removed when the bacteria were washed prior to their addition to the mucin-coated wells. To confirm this, mucin-coated wells were individually pretreated with the monosaccharides before the addition of strain D471. None of the sugars significantly affected bacterial adherence to mucin coated wells (not shown) suggesting that sialic acid inhibited adherence by interacting with the bacterial adhesin(s) rather than with the mucin.

Binding of the M6 protein to sialic acid on pharyngeal cell. As the treatment with sialic acid significantly decreased adherence of strain D471 to pharyngeal cells, we were interested in determining if the M protein was involved in this interaction. To this end, strain JRS75, the M-negative mutant of D471 (which differs only in its ability to produce the M protein) was used in the binding assays (FIG. 6A). As can be seen, the adherence of JRS75 to pharyngeal cells is not affected by sialic acid. The effect of sialic acid on D471 therefore, is most likely the result of the monosaccharide interacting with the M protein.

Sialylated linkages. 3′sialyllactose (3′SL) and the glycoproteins, fetuin and transferrin, were used as potential inhibitors in vitro to determine the effect of each compound on the adherence of streptococcal strain D471 to pharyngeal cells. Since these compounds contain different sialylated linkages, our hope was to determine if a specific linkage is important in the binding of the M6 protein to the Detroit cells. 3′SL contains sialic acid linked α2-3 to galactose while fetuin contains both α2-3 and α2-6 linkages (28), although according to the manufacturer (Sigma), the ratio of the two linkages has not been determined. Transferrin contains oligsaccharides that terminate only in α2-6 linked sialic acid (45).

FIG. 6B shows that 3′SL (1.7 mM) had no significant effect on adherence of strain D471 to the cultured pharyngeal cells. Fetuin (1.8 mM total sialic acid) was able to decrease adherence by approximately 30%. Even though the ratio of sialylated linkages in fetuin has not been determined, it is likely that this decrease in adherence was not the result of the α2-3 linked sialic acid since 3′SL had little effect on adherence. However, pretreatment of strain D471 with transferrin reduced adherence to cultured pharyngeal cells by approximately 90%. Since transferrin contains only α2-6 linked sialic acid, it seemed likely that this configuration was important in M protein interactions.

To further verify that sialic acid and not another component of transferrin was responsible for the decrease in adherence of strain D471, 6′sialyllactose (6′SL) (which contains sialic acid linked α2-6 to galactose) was used in inhibition assays. 6αSL (1.7 mM) was able to reduce binding of this strain to pharyngeal cells by approximately 85% (FIG. 6C). Because 6′sialyllactose and transferrin contain only α2-6 linked sialic acid, this sialylated linkage is directly implicated in the interaction of the M6 serotype with sialic acid-containing receptors on the Detroit 562 pharyngeal cell. Bacterial plate counts determined that none of the compounds tested effected the viabiilty of the organisms.

Effect of neuraminidase on streptococcal adherence to pharyngeal cells. Pharyngeal cell monolayers were treated with C. perfringens neuraminidase prior to being inoculated with streptococcal strain D471. This treatment decreased streptococcal adherence by approximately 80% (FIG. 6D) further implicating sialic acid in the adherence process. The enzyme from C. perfringens cleaves sialic acid linked α2-3 preferentially, but will also cleave sialic acid that is linked α2-6 and α2-8.

Identification of the sialylatedpharyngeal cell proteins that bind the M protein. The preceding experiments provide strong evidence that sialic acid on the surface of the pharyngeal cells plays an important role in the adherence of strain D471 through the M protein molecule. Our next goal was to identify the sialylated membrane proteins on pharyngeal cells and to determine which of these glycoproteins bind the M protein. To this end, a lectin from Tritrichomonas mobiliensis, which exclusively binds sialic acid, was used to probe blots of pharyngeal cell membrane proteins in parallel with duplicate blots probed with the purified M protein. This analysis revealed three pharyngeal cell proteins (65 kDa, 43 kDa, and 35 kDa) which bound the M protein as well as the sialic acid-specific lectin (FIG. 7). All three proteins are considered potential receptor molecules.

### Discussion

The sequence of events that lead to group A streptococcal infection of the upper respiratory mucosal tissue has not been fully elucidated, although it is clear that microbes which infect through mucosal surfaces (which account for >90% of all infections) share two initial goals. First, they must overcome the mucus layer that coats the mucosal epithelium and second, they must be able to attach to, and infect, the underlying target tissue. The results described here are the first to identify sialic acid as a receptor for group A streptococci, and this interaction lends insight into how streptococci reach the initial goals necessary to establish infection.

Mucus and in particular mucin, the major glycoprotein component of mucus, are presumably the first barriers that group A streptococci encounter upon entering the human upper respiratory tract. Mucins not only coat the pharyngeal mucosa, but are also similar in structure to cell surface glycoproteins (Ramphal, R., et al., Infect. Immun. 55:600-603, 1987). Hence, by defining the streptococcal interaction with mucin and the structural components involved, we may precisely identify potential pharyngeal receptors used in the adherence process.

Our results show that the N-terminal region of the M protein is the molecule responsible for binding streptococci to the sialic acid residues of mucin. This binding is in accord with previous studies on pathogens such as P. aeruginosa (Vishwanath, S., et al., Infect. Immun. 48:331-335, 1985), H. pylori (Tzouvelekis, L. S., et al., Infect. Immun. 59:4252-4254, 1991) and H. influenzae (Reddy, M. S., et al., Infect. Immun. 64:1477-1479, 1996) which also bind to the sialic acid residues on mucin, however the adhesins involved in these interactions have not been well characterized. Thus, sequence comparisons to identify common features between the M molecule and other bacterial mucin binding proteins were not possible.

Although the mechanism by which these particular bacterial proteins interact with sialic acid has not been elucidated, advances have been made in understanding the molecular basis for the binding of bacterial adhesins to other sialic acid containing molecules. A sialic acid binding motif has been identified in adhesins from E. coli and H. pylori (Evans, D. G., et al., J. Bacteriol. 175:674-683, 1993 and Jacobs, A. A. C., et al., Biochem. Biophys. Acta. 872:183-190, 1986). Close examination of the amino acid sequence of the M6 protein N-terminal region revealed no significant homology to this motif, suggesting that the interaction between the M protein and sialic acid is mediated by a different mechanism. One such mechanism has been proposed for the SspB polypeptide (formerly SSP-5) from S. gordonii, which binds to sialic acid on the salivary agglutinin glycoprotein (SAG), but does not contain the sialic acid binding motif (Demuth, D. R., et al., J. Biol. Chem. 265:7120-7126, 1990). Interestingly, the N-terminal portions of the SspB protein and the M protein are similar, not on the amino acid level, but rather due to similarities in the periodic distribution of hydrophobic amino acids found in these coiled-coil proteins (Jenkinson, H. F., et al., Molecular Microbiology 23:183-190, 1997) and the presence of amino acids with alpha-helical potential (Demuth, D. R., et al., J. Biol. Chem. 265:7120-7126, 1990). Thus, the conservation of secondary structure in the N-terminal regions of these two sialic acid binding proteins suggests that the coiled-coil structure may be involved in binding the monosaccharide (Demuth, D. R., et al., J. Biol. Chem. 265:7120-7126, 1990).

The terms sialic acid and N-acetylneuraminic acid are often used interchangeably, although it is important to make a distinction between the two. Sialic acid refers to a family of 9-carbon monosaccharides, of which N-acetylneuraminic acid is both the most common member and the metabolic precursor of a group of more than forty 9-carbon sugars (Varki, A., FASEB J. 11:248-255, 1997). The diversity of sialic acids lies in the various substitutions at different carbon positions, in addition to various linkages from carbon-2 to different underlying sugar chains. It is generally accepted that the recognition of a particular sialic acid is based on its particular structure, and that any substitution or change in linkage will also alter the recognition of a specific sialic acid (Varki, A., FASEB J. 11:248-255, 1997).

Since many epithelial membrane proteins contain sialic acid, and because the M protein has been shown to be important in the adherence process to epithelial cells (Fluckiger, U., et al., Infect. Immun. 66:974-979, 1999), we were prompted to investigate whether sialic acid, and more specifically, a particular sialylated linkage, are important in streptococcal adherence to pharyngeal cells. Two different approaches were taken to answer this question.

First, the role of sialic acid in M protein mediated adherence to pharyngeal cells was clear when exogenous sialic acid drastically decreased the binding of M protein-producing streptococci, but did not affect the adherence of an M-negative strain. Since many epithelial membrane proteins contain sialic acid, we ascertained that by identifying the sialylated linkage that is involved, we may hone in on the receptor(s) for the M protein. Previous reports have shown that various bacterial and viral adhesins have a preference for particular sialylated linkages. The S fimbriae from E. coli (Parkkinen, J., et al., Infect. Immun. 54:37-42, 1986), and adhesins from H. influenzae (Miller-Podraza, H., J., et al., Infect. Immun. 67:6309-6313, 1999) and H. pylori (Simon, P. M., et al., Infect. Immun. 65:750-757, 1997) bind to α2-3 linked sialic acid, whereas the influenza virus hemagglutinin binds specifically to α2-6 linked sialic acid (Barthelson, R., et al., Infect. Immun. 66:1439-1444, 1998). In addition, an adhesin from Streptococcus pneumoniae has been shown to bind to either α2-3 or α2-6 linked sialic acid (Barthelson, R., et al., Infect. Immun. 66:1439-1444, 1998). We found that 6′sialyllactose and transferrin, which contain only α2-6 linked neuraminic acid, interfered with the adherence of M-protein producing streptococci to pharyngeal cells, implicating this particular sialylated linkage in M protein recognition. Compounds containing sialic acid linked in other configurations did not affect adherence of the bacteria. It should be noted that our initial studies showed that the M protein binds to sialic acid on bovine submaxillary mucin, and structural studies on this type of mucin have determined that sialic acid is also linked by an α2-6 linkage (to N-acetyl galactosamine) (Davis, L., et al., Infect. Immun. 24:780-786, 1979).

Second, neuraminidase treatment of the pharyngeal cells decreased the adherence of the M protein-producing streptococci, further emphasizing the role of sialic acid in the adherence process. Although the enzyme from C. perfringens preferentially cleaves α2-3 linked sialic acid, α2-6 and α2-8 linked sialic acids are also enzyme substrates. It seems unlikely that the decrease in streptococcal adherence after neuraminidase treatment can be attributed to the cleavage of an α2-3 linked sialic acid, since 3′SL (which contains the preferred substrate, NeuAc α2-3Gal) failed to decrease streptococcal adherence in inhibition experiments. Thus, the decrease in adherence following neuraminidase treatment is likely due to cleavage of the second preferred substrate, α2-6 linked sialic acid.

Previous studies have confirmed that glycoconjugates found on the surface of the respiratory epithelium contain α2-6 linked sialic acid structures (Barthelson, R., et al., Infect. Immun. 66:1439-1444, 1998). Many transformed or immortalized cell lines do not necessarily display the same repertoire of carbohydrates on their surfaces as the native cells from which they are derived. However, the Detroit 562 pharyngeal cell line used in our studies has been reported to faithfully display the carbohydrate epitopes which are representative of the native cells (Barthelson, R., et al., Infect. Immun. 66:1439-1444, 1998). Thus, it is likely that the role of sialic acid in streptococcal adherence in in vitro assays using this cell line, may represent what occurs in vivo in the human host.

We have identified three pharyngeal cell membrane proteins that bind both the M protein and the sialic acid specific lectin from Tritrichomonas mobiliensis. Work is currently in progress to identify these molecules and to determine their roles in adherence. In addition, we recently reported the identification of the sialylated membrane bound mucin, MUC-1, on the surface of pharyngeal cells (P. A. Ryan, V. Pancholi, and V. A. Fischetti, 100th General Meeting, American Society for Microbiology, abstr. D-13, 2000). This molecule is also currently under investigation as a receptor molecule for the M protein.

An examination of the initial events that occur when group A streptococci enter the upper respiratory tract of the human host is of primary importance in our understanding of streptococcal colonization of pharyngeal tissue. To our knowledge, this is the first report on the interaction of S. pyogenes and mucin. A strong binding to mucin in the upper respiratory tract of humans would seem to be counterproductive to streptococci in terms of its ability to initiate infection. However, if the mucin of certain individuals was not sialylated or did not contain the sialylated linkage that we show here is necessary for streptococcal binding, the bacteria would not bind to mucin and thus avoid efficient clearance from the airways. Work is underway to analyze the sialic acid content and the sialylation patterns of mucins from various individuals, including children, so that we may better understand the role these mucins play in streptococcal colonization.

Sialic acid is also important in another streptococcal interaction that directly relates to the colonization process, and that is in the adherence of streptococci to pharyngeal cells. Furthermore, we have shown that the structural configuration of sialic acid is important in this M protein-based recognition. It should be noted that many of the cell surface glycoproteins that have been implicated in streptococcal adherence, such as the integrins (Ozeri, V., et al., Mol. Microbiol. 30:625-637, 1998), fibronectin (Courtney, H. S., et al., Infect. Immun. 53:454-459, 1986; Okada, N., et al., J. Biol. Chem. 272:26978-26984, 1998 and Rocha, C. and V. A. Fischetti. Unpublished data. 1999), and plasminogen (Pancholi, V., et al., J. Biol. Chem. 273:14503-14515, 1998), are all sialylated glycoproteins.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate, and are provided for description.

Patents, patent applications, publications, product descriptions, and protocols are cited throughout this application, the disclosures of which are incorporated herein by reference in their entireties for all purposes.

We claim:

1. A method for binding an isolated group A streptococcal M protein to an isolated mucin which method comprises contacting the M protein with the mucin.

2. The method of claim 1 wherein the mucin is bound to a solid substrate.

3. The method of claim 1 wherein the group A streptococcal cell is strain D471.

4. The method of claim 1 wherein the M protein is bound to a solid substrate.

5. The method of claim 4 wherein the M protein is bound to a membrane.

6. The method of claim 1 wherein the mucin is derived from a pharyngeal cell.

7. The method of claim 6 wherein the pharyngeal cell is strain Detroit 562.

8. A method for preventing adhesion or invasion of a cell comprising mucin on an outer surface by a group A streptococcal cell comprising M protein on an outer surface comprising the step of inhibiting contact between the mucin and the M protein.

9. The method of claim 8 which comprises contacting the M protein with a substance which is selected from the group consisting of sialic acid, transferrin and an anti-M protein antibody.

10. The method of claim 8 which comprises contacting the mucin with a substance which is selected from the group consisting of neuraminidase, a soluble form of M protein and a soluble form of an N-terminal mucin binding fragment of M protein.

11. The method of claim 10 wherein the substance is neuraminidase and the neuraminidase is C. perfringens neuraminidase.

12. The method of claim 8 wherein said group A streptococcal cell is group A streptococcal strain D471.

13. The method of claim 8 wherein said cell comprising mucin on an outer surface is a pharyngeal cell.

14. The method of claim 13 wherein said pharyngeal cell comprises a Detroit 562 cell.

15. The method of claim 8 wherein the group A streptococcal cell and the cell comprising mucin on an outer surface are located within a subject's body.

16. A method for treating or preventing a group A streptococcal infection in a subject comprising administering to the subject one or more substances selected from the group consisting of a sialic acid, 3′sialyllactose, 6′sialyllactose, neuraminidase, transferrin, an antibody, 10B6 antibody, 10A11 antibody, a soluble form of M protein and a soluble form of an N-terminal mucin binding fragment of M-protein.

17. The method of claim 16 wherein the group A streptococcal infection is caused by strain D471.

18. The method of claim 16 wherein the infection is located at a site in the subject's body which comprises a cell which comprises mucin on an outer surface.

19. The method of claim 18 wherein the cell is a pharyngeal cell.

20. A method for identifying a candidate substance which inhibits binding between a group A streptococcal M protein and mucin which method comprises determining if M protein and mucin, contacted with the candidate substance, bind to each other to the same extent as they do in the absence of the candidate substance and selecting the candidate substance if it is correlated with reducing binding between the M protein and the mucin.

21. The method of claim 20 wherein the M protein is isolated.

22. The method of claim 20 wherein the mucin is isolated.

23. The method of claim 20 wherein the M protein is associated with a group A streptococcal cell.

24. The method of claim 20 wherein the mucin is associated with a mucin producing cell.

25. The method of claim 20 wherein the mucin is pharyngeal cell mucin.

26. The method of claim 25 wherein the pharyngeal cell is a Detroit 562 cell.

27. The method of claim 20 wherein the M protein is derived from group A streptococcal strain D471.

28. The method of claim 20 wherein the candidate substance is selected from the group consisting of an antibody, a sialic acid saccharide, a polypeptide, a neuraminidase, a sialic acid specific lectin and a small molecule.

29. A method for preventing or treating a group A streptococcal infection in a subject comprising administering a vaccine comprising group A streptococcal M protein or a fragment thereof or a nucleic acid comprising a gene which encodes group A streptococcal M protein or a fragment thereof to the subject.

30. The method of claim 29 wherein the group A streptococcal M protein fragment is an N-terminal, 30 kDa fragment of group A streptococcal M protein.

31. The method of claim 29 wherein the group A streptococcal M protein is derived from strain D471.

32. The method of claim 29 wherein the vaccine comprises a nucleic acid and the nucleic acid comprises plasmid DNA.

33. The method of claim 29 wherein vaccine comprises a nucleic acid and the gene is operatively associated with a promoter.

34. The method of claim 33 wherein the promoter is selected from the group consisting of an RSV promoter and a CMV promoter.

35. A polypeptide comprising an amino-terminal, 30 kDa fragment of group A streptococcal M protein which binds to mucin.

36. A vaccine comprising a group A streptococcal M protein or a fragment thereof or a nucleic acid comprising a gene which encodes group A streptococcal M protein or a fragment thereof and a pharmaceutically acceptable carrier.

37. The vaccine of claim 36 wherein the vaccine comprises a fragment of group A streptococcal M protein or a nucleic acid which encodes the fragment and the fragment comprises an amino-terminal, 30 kDa fragment of group A streptococcal M protein which binds to mucin.

38. The vaccine of claim 36 comprising a nucleic acid which comprises a gene which encodes group A streptococcal M protein or a fragment thereof wherein the gene is operatively associated with a promoter.

39. The vaccine of claim 38 wherein the promoter is selected from the group consisting of a CMV promoter and an RSV promoter.

40. The vaccine of claim 36 wherein the carrier is immunogenic.

41. A Pharmaceutical composition comprising apharmaceutically acceptable carrier and one or more members selected from the group consisting of sialic acid, 3′-sialyllactose, 6′-sialyllactose, neuraminidase, transferrin, an anti-M protein antibody, 10A11 antibody, 10B6 antibody, a soluble form of M protein and a soluble form of an N-terminal mucin binding fragment of M protein.


SEQUENCE LISTING (2 sequences)

SEQ ID NO: 1 - DNA, 2111 bp, Streptococcus pyogenes
gatctagaagagattgaaaaacagtatgatgtgatcgtgacagatgttatggtaggaaaaagcgatgagttagaaattttctttttctacaaaatgattccagaagcgattattgacaagctcaatgtgtttttaaacatcagctttgcagacagcttggccactagagcaaacccatccaagaaccccttggactttcatcgcaaagagcttacctttacccactccccccaacaacgttttgcacgccccccccgcacgaagttagacagcctagccgcagaaactcaaaaacagattcatcattaatagcatttaggtcaaaagtggcaaaagctaaaaaagctggtctttaccttttggcttttattatttacaatacaattattagagttaaaccctgaaaatgagggtttttcctaaaaaatgataacataaggagcataaaaatggctaaaaataacacgaatagacactattcgcttagaaaattaaaaaaaggtactgcatcagtagcagtggctttgagtgtaataggggcaggattagttgtcaatactaatgaagttagtgcaagagtgtttcctagggggacggtagaaaacccggacaaagcacgagaacttcttaacaagtatgacgtagagaactctatgttacaagctaataatgacaagttaacaactgagaataataacttaacagatcagaataaaaacttaacaactgagaataaaaacttaacagatcagaataaaaacttaacaactgagaataaaaacttaacagatcagaataaaaacttaacaactgagaataaggagttaaaagctgaggagaataggttaacaactgagaataaagggttaactaaaaagttgagtgaagctgaagaagaagcagcaaataaagagcgagaaaataaagaagccattggtacccttaaaaaaaccttggatgagacagtaaaagataaaattgctaaggagcaagaaagtaaagaaaccattggtacccttaaaaaaaccttggatgagacagtaaaagataaaattgctaaggagcaagaaagtaaagaaaccattggtacccttaaaaaaaccttggatgagacagtaaaagataaaattgctaaggagcaagaaagtaaagaaaccattggtacccttaaaaaaatcttggatgagacagtaaaagataaaattgcgagagagcaaaaaagtaaacaagacattggtgcccttaaacaagaattagctaaaaaagatgaaggaaacaaagtttcagaagcaagccgtaagggtcttcgccgtgacttggacgcatcacgtgaagctaagaaacaggttgaaaaagatttagcaaacttgactgctgaacttgataaggttaaagaagaaaaacaaatctcagacgcaagccgtcaaggtcttcgccgtgacttggacgcatcacgtgaagctaagaaacaagttgaaaaagctttagaagaagcaaacagcaaattagctgctcttgaaaaacttaacaaagagcttgaagaaagcaagaaattaacagaaaaagaaaaagctgagctacaagcaaaacttgaagcagaagcaaaagcactcaaagaacaattagcgaaacaagctgaagaacttgcaaaactaagagctggaaaagcatcagactcacaaacccctgatgcaaaaccaggaaacaaagttgttccaggtaaaggtcaagcaccacaagcaggtacaaaacctaaccaaaacaaagcaccaatgaaggaaactaagagacagttaccatcaacaggtgaaacagctaacccattcttcacagcggcagcccttactgttatggcaacagctggagtagcagcagttgtaaaacgcaaagaagaaaactaagctatcactttgtaatactgagtgaacatcaagagagaaccagtcggttctctcttttatgtatagaagaatgagattaaggaggtcacaaactaaacaactcttaaaaagctgacctttactaataatcgtcttttttttataataaagatgttaataatataattgataaatgagatacatttaatgattatgacaaaagcaagaaaa

SEQ ID NO: 2 - protein, 483 aa, Streptococcus pyogenes
MAKNNTNRHYSLRKLKKGTASVAVALSVIGAGLVVNTNEVSARVFPRGTVENPDKARELLNKYDVENSMLQANNDKLTTENNNLTDQNKNLTTENKNLTDQNKNLTTENKNLTDQNKNLTTENKELKAEENRLTTENKGLTKKLSEAEEEAANKERENKEAIGTLKKTLDETVKDKIAKEQESKETIGTLKKTLDETVKDKIAKEQESKETIGTLKKTLDETVKDKIAKEQESKETIGTLKKILDETVKDKIAREQKSKQDIGALKQELAKKDEGNKVSEASRKGLRRDLDASREAKKQVEKDLANLTAELDKVKEEKQISDASRQGLRRDLDASREAKKQVEKALEEANSKLAALEKLNKELEESKKLTEKEKAELQAKLEAEAKALKEQLAKQAEELAKLRAGKASDSQTPDAKPGNKVVPGKGQAPQAGTKPNQNKAPMKETKRQLPSTGETANPFFTAAALTVMATAGVAAVVKRKEEN